(19) United States
(12) Patent Application Publication
(10) Pub. No.: US 2003/0114382 A1
(43) Pub. Date: Jun. 19, 2003

(54) Glycogen synthase kinase function in endothelial cells

(76) Inventor: Kenneth Walsh, Carlisle, MA (US)

Correspondence Address: WOLF GREENFIELD & SACKS, PC, FEDERAL RESERVE PLAZA, 600 ATLANTIC AVENUE, BOSTON, MA, 02210-2211, US

(21) Appl. No.: 10/278,759

(22) Filed: Oct. 23, 2002

Related U.S. Application Data
(60) Provisional application No. 60/350,160, filed on Oct. 29, 2001.
(60) Provisional application No. 60/337,905, filed on Nov. 13, 2001.

(51) Int. Cl. A61K 48/00; A61K 38/17; G01N 33/53; C12Q 1/48

(52) U.S. Cl. 514/12; 514/44; 435/15; 435/7.1

(57) ABSTRACT

This invention relates to methods and compositions for modulating endothelial cell viability, endothelial cell migration, and angiogenesis. In particular the invention relates to GSK3 molecules, agents that modify the kinase activity of these molecules, and use of the foregoing in modulating the endothelial cell viability, endothelial cell migration, and angiogenesis.

### RELATED APPLICATIONS

This application claims priority under 35 U.S.C. §119 to U.S. provisional application serial No. 60/350,160, filed Oct. 29, 2001, the entire disclosure of which is incorporated herein by reference. This application also claims priority under 35 U.S.C. §119 to U.S. provisional application serial No. 60/337,905, filed Nov. 13, 2001, the entire disclosure of which is incorporated herein by reference.

### GOVERNMENT SUPPORT

This work was supported by National Institutes of Health grants AR40197, HL50692, AG15052 and AG17241. The government may have rights to the inventions disclosed herein.

### FIELD OF THE INVENTION

This invention relates to methods and compositions for modulating endothelial cell viability, endothelial cell migration, and angiogenesis. In particular the invention relates to GSK3 molecules, agents that modify the kinase activity of these molecules, and use of the foregoing in modulating the foregoing activities.

### BACKGROUND OF THE INVENTION

Glycogen synthase kinase-3 (GSK3) is a highly conserved and ubiquitously expressed serine/threonine kinase that phosphorylates proteins containing clustered serine or threonine residues that are separated by 4 amino acids (1-4). GSK3a and GSK3β are encoded by different genes, and they are 85% homologous in their amino acid sequence. Both isoforms have similar substrate specificity and are regulated in parallel in response to growth factors (1, 5, 6). Disruption of the GSK3β gene in mice results in embryonic lethality, indicating that GSK3 α cannot completely substitute for a loss of GSK3β (7). Although GSK3 was originally identified as a kinase that phosphorylates glycogen synthase (8), subsequent studies have demonstrated that it has broader range of substrates including β-catenin (9), tau (10), myelin basic protein (11), cyclin D1 (12), GATA4 (13), c-jun (14), c-myc (15), CREB (16), initiation factor eIF2B (17), heat shock factor-1 (18), and p53 (19). Through the phosphorylation of this diverse set of substrates, GSK3 regulates embryonic development and proliferative responses in adult tissues, and is implicated in several human disease states including tumorigenesis, Alzheimer's disease, and diabetes (20).

GSK3 signaling reportedly is inactivated in cells that are stimulated by mitogens. Growth factor-induced inactivation involves phosphorylation of N-terminal serine residue (Ser 21 for GSK3α and Ser 9 for GSK3β). This phosphorylation can be mediated by several upstream protein kinases including p90RSK, p70S6K, integrin-linked kinase, Akt, and protein kinase A (PKA) (5, 6, 21-24). GSK3 is also regulated by Wnt signaling during embryonic development, leading to the specification of cell fate (2, 25). Mitogenic and Wnt signaling differentially regulate GSK, and this may elicit distinct downstream responses (1, 20, 26).

Relatively little is known about the role of GSK3 signaling in the cardiovascular system. Recently, two studies have reported that GSK3β signaling inhibits cardiac myocyte hypertrophy, an effect that may be mediated through its regulation of NFAT or GATA4 transcription factors (13, 27). It has also been reported that GSK3β promotes apoptosis in cultured vascular smooth muscle cells (28).

### SUMMARY OF THE INVENTION

The invention is based, in part, on the discovery of a function for GSK3 in endothelial cells (EC) and the discovery of the role played by GSK3 in blood vessel formation. In view of these discoveries, methods and compositions for modulating angiogenesis by modulating GSK3 are provided. The methods involve administering to a subject a GSK3 molecule or an agent which modulates the activity of a GSK3 molecule. The methods and compositions are administered in accordance with standard procedures such as those described in clinical textbooks.

According to one aspect of the invention, a method for inhibiting angiogenesis is provided. The method involves administering to a subject in need of such treatment an angiogenesis inhibitor in an amount effective to inhibit angiogenesis in the subject. The angiogenesis inhibitor may be an “active GSK3 molecule” or a “GSK3 kinase activator”.

As used herein, an “active GSK3 molecule” refers to a GSK3 molecule that has a protein kinase activity, i.e., the GSK3 molecule has an enzymatic activity that permits it to phosphorylate a protein substrate. Preferably, the GSK3 molecule is a GSK3 nucleic acid molecule having SEQ ID NOS: 1, 9, 11, or 13 (GSK3 nucleic acid sequences) or a GSK3 polypeptide molecule having SEQ ID NOS: 2, 3, 4, 5, 6, 7, 8, 10, 12, or 14 (GSK3 polypeptide sequences). The corresponding GenBank Accession Nos. and a brief description of these sequences is provided below in Table 1 showing the Abbreviated Sequence Listing. Thus, a GSK3 molecule as used herein, may be an active GSK3 nucleic acid molecule (i.e., a nucleic acid that encodes an active GSK3 polypeptide) or the encoded active GSK3 polypeptide molecule. Additionally, the active GSK3 molecule may be a nucleic acid or encoded polypeptide that is constitutively active, i.e., the sequence of the GSK3 nucleic acid molecule or of the GSK3 polypeptide molecule has been altered to prevent phosphorylation of the GSK3 polypeptide molecule (e.g., by an Akt molecule). Although not wishing to be bound to any particular theory or mechanism, it is believed that the naturally occurring active GSK3 molecule is not phosphorylated and that phosphorylation of GSK3 (e.g., by an Akt molecule) inhibits GSK3 kinase activity. Akt molecules are described in U.S. Ser. No. 09/408,905, entitled: AKT COMPOSITIONS FOR ENHANCING SURVIVAL OF CELLS, filed Sep. 29, 1999, the entire contents of which are incorporated herein by reference. As noted in the cited application, Akt molecules include wild-type Akt molecules and constitutively-active Akt molecules.

Alternatively, the angiogenesis inhibitor may be a “GSK3 kinase activator”. As used herein, a “GSK3 kinase activator” refers to a molecule that is capable of mediating a transition from an inactive GSK3 molecule (e.g., a GSK3 molecule that has been phosphorylated by an Akt molecule) into an active GSK3 molecule having a protein kinase activity. GSK3 kinase activators may be identified in screening assays which identify agents which mediate the transition from an inactive to an active GSK3 molecule (e.g., by observing an inhibition of an endothelial cell activity (viability, migration, angiogenesis) in the presence of the putative GSK3 kinase activator).

Subjects in need of inhibiting angiogenesis include subjects diagnosed as having a condition associated with undesirable endothelial cell proliferation (e.g., a cancer involving endothelial cells), or a predisposition to a condition associated with undesirable endothelial cell proliferation. The subject may or may not have a condition calling for treatment with an Akt inhibitor or an agent that downregulates expression of an Akt molecule in the subject. In some embodiments, the subject does not have a condition otherwise calling for treatment with an active GSK3 molecule prescribed or administered to the subject for treatment or as part of a clinical trial.

In some embodiments, the angiogenesis inhibitor is administered acutely to prevent future or further angiogenesis (e.g., to prevent further angiogenesis associated with a solid tumor). In some preferred embodiments, acute administration of the angiogenesis inhibitor is to and/or in the area adjacent to a solid tumor.

S According to another aspect of the invention, a method for enhancing angiogenesis is provided. The method involves administering to a subject in need of such treatment an “angiogenesis promoter” in an amount effective to enhance angiogenesis in the subject. The angiogenesis promoter is an “inactive GSK3 molecule” or a “GSK3 kinase inhibitor”.

As used herein, an “inactive GSK3 molecule” refers to a GSK molecule which has reduced or no kinase activity compared to a wild-type GSK3 molecule (e.g., compared to a wild-type human GSK3 molecule such as SEQ ID NO: 1 or 2). Inactive GSK3 molecules include nucleic acid molecules and polypeptide molecules. In certain embodiments, the inactive GSK3 molecule is a are GSK3 polypeptide that has been phosphorylated (e.g., by an Akt molecule).

As used herein, a GSK3 kinase inhibitor refers to a molecule which is capable of mediating a transition from an active GSK3 molecule which has a kinase activity to an inactive GSK3 molecule having no or reduced protein kinase activity. GSK3 kinase inhibitors may be identified in screening assays which identify agents which mediate the transition from an active to an inactive GSK3 molecule (e.g., by observing an enhancement of an endothelial cell activity such as viability, migration, or angiogenesis in the presence of the putative GSK3 kinase inhibitor). In certain embodiments, GSK3 kinase inhibitors exclude one or more known protein kinases (e.g., Akt) which phosphorylate and, thereby, enhance GSK3 kinase activity.

Subjects in need of enhancing angiogenesis include subjects with myocardial infarction, ischemia-reperfusion injury, dilated cardiomyopathy, and conductive system disorders. In some embodiments, a growth factor may be co-administered. In some embodiments, Insulin-like Growth Factor-1 (IGF-1) is the growth factor preferably utilized. In some embodiments, the angiogenesis promoter is administered acutely to prevent future or further tissue damage (e.g., cardiac tissue necrosis). In some embodiments, acute administration of the angiogenesis promoter is to the apical and anterolateral free wall of the heart. In these and or other embodiments, the subject may or may not have a condition calling for treatment with an Akt molecule or molecule that upregulates expression of an Akt molecule in the subject. In some embodiments, the subject does not have a condition calling for treatment with an angiogenesis promoter of the invention (i.e., an inactive GSK3 molecule and/or a GSK3 kinase inhibitor have not been prescribed or administered to the subject for treatment or as part of a clinical trial).

In some embodiments, the invention involves co-administration of at least one anti-arteriosclerotic agent used in the treatment of an arteriosclerotic condition, with at least one angiogenesis promoter. In preferred embodiments, the anti-arteriosclerotic agent is selected from the group consisting of a 3-hydroxy-3-methylglutaryl-coenzyme A (HMG-CoA) reductase inhibitor, a diuretic, an antiadrenergic agent, a vasodilator, a calcium channel antagonist, an angiotensin-converting enzyme (ACE) inhibitor, an angiotensin II antagonist, and a clot dissolver together with an angiogenesis promoter to treat myocardial infarction and inhibit endothelial cell death (particularly, vascular endothelial cell death) in the subject.

According to another aspect of the invention, a method for inhibiting an endothelial cell activity is provided. The method involves contacting an endothelial cell with an angiogenesis inhibitor under conditions that permit the angiogenesis inhibitor to inhibit an endothelial cell activity. In some embodiments, the method involves contacting the endothelial cell with an angiogenesis inhibitor under conditions that permit the angiogenesis inhibitor to enter the endothelial cell in an effective amount to inhibit an endothelial cell activity. Exemplary endothelial cell activities include endothelial cell viability, endothelial cell migration, and angiogenesis. The contacting of the endothelial cell with the angiogenesis inhibitor may be performed in vitro or in vivo. The angiogenesis inhibitor is as defined above and includes active GSK3 molecules (e.g., an active GSK3 nucleic acid molecule, an active GSK3 polypeptide molecule), as well as GSK3 kinase activators.

According to yet another aspect of the invention, a method for enhancing an endothelial cell activity is provided. The method involves contacting an endothelial cell with an angiogenesis promoter under conditions that permit the angiogenesis promoter to enhance an endothelial cell activity (e.g., endothelial cell viability, endothelial cell migration, angiogenesis). In some embodiments, the method involves contacting the endothelial cell with an angiogenesis promoter under conditions that permit the angiogenesis promoter to enter the endothelial cell in an effective amount to enhance endothelial cell activity. The angiogenesis promoter is as defined above and includes inactive GSK3 molecules (e.g., an inactive GSK3 nucleic acid molecule, an inactive GSK3 polypeptide molecule), as well as GSK3 kinase inhibitors. The method may be performed in vitro or in vivo.

According to a further aspect of the invention, a method for inhibiting apoptotic cell death of an endothelial cell (e.g., vascular endothelial cell) is provided. The method involves contacting an angiogenesis promoter with an endothelial cell wherein the angiogenesis promoter is present in an amount effective to inhibit apoptotic cell death of the endothelial cell. In some embodiments, the method involves contacting the endothelial cell with an angiogenesis promoter under conditions that permit the angiogenesis promoter to enter the endothelial cell in an effective amount to inhibit apoptotic cell death. The angiogenesis promoter is as defined above and includes inactive GSK3 molecules (e.g., an inactive GSK3 nucleic acid molecule, an inactive GSK3 polypeptide molecule), as well as GSK3 kinase inhibitors. The method may be performed in vitro or in vivo. In some embodiments, the endothelial cell is part of a tissue or an organ to be transplanted. In these and/or other embodiments, contacting of an angiogenesis promoter with an endothelial cell may involve acute administration of the angiogenesis promoter. In some embodiments, contacting of an angiogenesis promoter with an endothelial cell involves prophylactic administration of the angiogenesis promoter to a subject. Optionally, a growth factor (e.g., a Vascular Endothelial Growth Factor (VEGF)) is co-administered.

According to two other aspects of the invention, a composition including an isolated active GSK3 nucleic acid molecule or a composition including an isolated inactive GSK3 nucleic acid molecule is provided. The isolated active GSK3 nucleic acid molecule or the isolated inactive GSK3 nucleic acid molecule is operably linked to a gene expression sequence which permits expression of the active GSK3 nucleic acid molecule or of the inactive GSK3 nucleic acid molecule in an endothelial cell (e.g., vascular endothelial cell). In some embodiments, the isolated GSK3 nucleic acid molecule is contained in an appropriate expression vector (e.g., adenoviral vector, modified adenoviral vector, retroviral vector, plasmid, liposome) to more efficiently genetically modify the targeted cell and achieve expression of active GSK3 molecule or inactive GSK3 molecule in the targeted cell. In some embodiments, the vector is an adenoviral vector.

According to yet another aspect of the invention, a method of screening for a GSK3 kinase modulator (activator or inhibitor) that modulates (enhances or inhibits) an endothelial cell activity is provided. The method involves: (a) contacting a test molecule with an endothelial cell under conditions to permit entry of the test molecule into the cell; and (b) determining whether the test molecule modulates an endothelial cell activity (e.g., viability, migration, angiogenesis).

An increase in an endothelial cell activity in the presence of the test molecule indicates that the test molecule is a GSK3 kinase inhibitor; a decrease in an endothelial cell activity in the presence of the test molecule indicates that the test molecule is a GSK3 kinase activator. Test molecules may be members of a library of molecules such as a phage display library or a chemical combinatorial library. The screening method may be performed in vitro or in vivo (e.g., an animal model).

In yet another aspect of the invention, a method for treating a condition associated with increased apoptotic cell death of vascular endothelial cells is provided. The method involves administering to a subject in need of such treatment an angiogenesis promoter in an amount effective to inhibit increased apoptotic cell death of vascular endothelial cells. The angiogenesis promoter is as defined above and includes inactive GSK3 molecules (e.g., an inactive GSK3 nucleic acid molecule, an inactive GSK3 polypeptide molecule), as well as GSK3 kinase inhibitors.

In one embodiment, constitutively inactive GSK3 molecules are utilized. In certain embodiments, the condition is characterized by lesions of a blood vessel wall. Examples of lesions of a blood vessel wall include traumatic injury such as, for example, following angioplasty or bypass grafting transplantation where the trauma could induce intimal hyperplasia. In some embodiments, lesions of a blood vessel wall (also known as endothelial cell dysfunction) are associated with hyperlipidemic subjects. In some embodiments, the angiogenesis promoter is administered acutely to prevent future or further tissue damage (e.g., endothelial cell dysfunction).

According to a further aspect of the invention, a pharmaceutical composition that includes any of the foregoing isolated human GSK3 molecules or agents which modulate the activity of these GSK3 molecules, in a pharmaceutically effective amount to modulate an endothelial cell activity, and a pharmaceutically acceptable carrier, is also provided. Methods for preparing such pharmaceutical compositions are also provided.

Such methods involve placing any of the foregoing isolated human GSK3 molecules or agents which modulate the activity of these GSK3 molecules, in a pharmaceutically effective amount to modulate an endothelial cell activity, in a pharmaceutically acceptable carrier. GSK3 nucleic acid molecules including vectors, as well as additional agents that may be included in the pharmaceutical compositions, are as described above.

These and other aspects of the invention, as well as various advantages and utilities, will be more apparent with reference to the detailed description.

### BRIEF DESCRIPTION OF THE DRAWINGS

FIG. 1. Multiple signaling pathways modulate GSK3β phosphorylation in EC. (A, B) Subconfluent HUVECs that were serum-starved for 15 hours and then stimulated with FBS 10% for 1 hour with or without 1 hour-pretreatment of the indicated agents (DMSO (dimethylsulfoxide), LY (LY294002), PD (PD98059), SB (SB203580), PKC-1 (protein kinase C inhibitor), BIM (bisindolylmaleimide), PKA-I (protein kinase A inhibitor), 8-Br-cAMP (8-bromo cAMP)). Immunoblot analyses were done for phospho-GSK, total GSK, phosphoAkt, and α-tubulin. (C) Modulation of GSK3β signaling by adenovirus-mediated gene transfer. Immunoblot analysis of subconfluent HUVECs in EGM with serum 2% at 1 and 2 days after infection with adenovirus at 50 MOI. Transduction of the constitutively active, nonphosphorylatable GSK3β-S9A decreases phosphorylated GSK3β, whereas catalytically inactive GSK3β-KM promotes phosphorylation. Only GSK3β constructs have hemagglutinin-tag (HA).

FIG. 2. GSK3β signaling controls EC chemotaxis toward angiogenic growth factors. (A) Low power field (X100) microscopic view of Giemsa-stained blue EC migrating through the filter toward VEGF (50 ng/ml) or basic FGF (25 ng/ml). HUVECs were infected with adenovirus at 50 MOI for 1 day in the presence of serum and then serum-starved for 5 hours before chemotaxis assay. (B) Quantitative data of Giemsa-stained EC that migrated through filter toward growth factor. Cells were counted in 3 randomly chosen low-power fields per well. Each experiment was performed in duplicate and 4 separate experiments were performed. (* p<0.01 between β-gal and GSK-S9A, n=8) (C) WST-1 assays of mitochondrial function 2 days after transduction with the indicated adenoviral vector at 25 or 50 MOI in the presence of serum. There is no significant difference between three experimental groups (n=12).

FIG. 3. GSK3β signaling controls HAoSMC chemotaxis towards growth factors. (A) Quantitative data of Giemsa-stained smooth muscle cells that migrated through filter toward PDGF-BB (50 ng/ml) or basic FGF (50 ng/ml). HAoSMCs were infected with adenovirus at 100 MOI for 1 day before the chemotaxis assay. Migrating cells were counted in 3 randomly chosen low-power fields per well. Each experiment was performed in duplicate and 4 separate experiments were performed. (* p<0.01 between β-gal and GSK-S9A, or GSK-KM, n=8) (B) Mitochondrial function in HAoSMC was assessed using WST-1 assay under the conditions employed by the chemotaxis assay. There was no significant difference of cell viability among three groups at 2 days after infection with adenovirus at 100 MOI (n=12).

FIG. 4. Effects of GSK3β signaling on DNA content and nuclear morphology under conditions of prolonged serum-deprivation in HUVEC. (A) FACS analysis of DNA content in propidium iodide-stained HUVEC after transduction with the indicated adenoviral vectors (100 MOI) and incubation in serum-free media for 4 days. (* p<0.01 between β-gal and GSK-KM, #p<0.05 β-gal and GSK-S9A, n=5) (B) Microscopic analysis of Hoechst 33342-stained HUVECs at 4 days after transduction with the indicated adenoviral vector (50 MOI) and incubation in serum-free media for 4 days. Three separate experiments were performed in quadruplicate. (* p<0.01 between β-gal and GSK-KM, #p<0.05 β-gal and GSK-S9A, n=12)

FIG. 5. Effects of GSK3β signaling on membrane phospholipid asymmetry and mitochondrial function under conditions of prolonged serum-deprivation in HUVEC. (A, upper panel) Phase contrast microscopic view of HUVECs transduced with the indicated adenoviral vectors (100 MOI) and incubated for 2 days in serum-free media. (A, lower panel) Fluorescent microscopic view of the cells double-strained with annexin V (green) and propidium iodide (red). Green bodies are HUVECs at the early stage of apoptosis. Red ones are necrotic cells. (B) Frequencies of annexin V-positive cells in the 3 experimental groups. (*p<0.01 between β-gal and GSK-KM, or β-gal and GSK-S9A, n=12) (C) WST-1 assay of mitochondrial function in HUVEC cultures transduced with the indicated adenoviral vectors (100 MOI) and incubated in the absence of serum for 4 days. (* p<0.01 between β-gal and GSK-S9A or GSK-KM, n=12).

FIG. 6. Effect of GSK3β signaling on EC anoikis. (A) Time-dependent decreases in Akt and GSK3β phosphorylation upon detachment of HUVEC from culture plate. Immunoblot analysis of HUVEC that were harvested at different times following transfer to polyHEMA-coated petri dishes which do not permit cellular attachment. (B) Representative phase contrast and fluorescent microscopic view of HUVECs that were infected with the indicated adenoviral vectors overnight prior to transfer to polyHEMA-coated plates for 1 day. Cells were double-stained with annexin V and propidium iodide. (C) Representative phase contrast microscopic views of HUVECs that were deprived of anchorage attachment for 1 day and then transferred to a standard cell culture plate where they were permitted to reattach for 1 or 2 days. (D) Quantitative data corresponding to assays in B and C. (* p<0.01 between β-gal and GSK-KM, or β-gal and GSK-S9A, n=12)

FIG. 7. Role of GSK3β signaling in a matrigel plug assay of angiogenesis in mice. Matrigel containing bFGF and the indicated adenoviral vector was injected subcutaneously in mice. After sacrifice plugs were excised and immunostained for the GSK3β transgene protein expression with anti-HA antigen antibodies (A, upper panels). Adjacent sections were stained for infiltrating EC using anti-CD31 antibody (A, middle panels). Sections were also stained for alkaline phosphatase activity to assess capillary infiltration (A, bottom panel). (B) Quantitative assessment of alkaline phosphatase-positive cells within the matrigel plugs. Three randomly chosen microscopic fields were counted from each section analyzed. Twelve sections from 3 animals were analyzed for each experimental group. Data were presented as the percentage of positive cells relative to the β-galactosidase-transduced group. (* p<0.01 between β-gal and GSK-KM, or β-gal and GSK-S9A, n=12). The matrigel plug and excised abdominal muscle (Abd Mus) is indicated in some photomicrographs.

TABLE 1
Abbreviated Sequence Listing

| SEQ ID NO: | DESCRIPTION |
|---|---|
| 1 | Human GSK3β cDNA (SEQ ID NO:1 nucleic acid sequence from U.S. |
| | 6,248,559 which is incorporated herein by reference) |
| 2 | Human GSK3β polypeptide (U.S. 6,248,559 SEQ ID NO:1 amino acid sequence) |
| 3 | Human GSK3α polypeptide (GenBank Accession No.XP_029918) |
| 4 | Human GSK3α polypeptide (GenBank Accession No.NP_063937) |
| 5 | Human GSKTβ polypeptide (GenBank Accession No.P49840) |
| 6 | Human G5K3β polypeptide (GenBank Accession No.P49841) |
| 7 | Human G5K3β polypeptide (GenBank Accession No.S53324) |
| 8 | Human GSK3β polypeptide (GenBank Accession No.NP_002084) |
| 9 | Human GSK3α cDNA (GenBank Accession No.XM_029918) |
| 10 | Human GSK3α polypeptide (GenBank Accession No.XM_029918) |
| 11 | Human GSK3α cDNA (GenBank Accession No.NM_019884) |
| 12 | Human GSK3α polypeptide (GenBank Accession No.NM_019884) |
| 13 | Human GSK3β mRNA (GenBank Accession No.NM_002093) |
| 14 | Human GSK3β polypeptide (GenBank Accession No.NM_002093) |

### DETAILED DESCRIPTION OF THE INVENTION

The invention is based, in part, on the discovery of a function for GSK3 in endothelial cells (EC) and the discovery of the role played by GSK3 in blood vessel formation. In view of these discoveries, methods and compositions for modulating angiogenesis by modulating GSK3 are provided. The methods involve administering to a subject a GSK3 molecule or an agent which modulates GSK3 kinase activity. The methods and compositions are administered in accordance with standard procedures such as those described in clinical textbooks. Additionally, methods for using these molecules in vivo or in vitro for the purpose of inhibiting apoptotic cell death and methods for treating conditions associated with such cell death are also provided.

The human GSK3 gene has been isolated and sequenced. See Table 1 of Abbreviated Sequence Listing presented above for the Genbank Accession Nos. for the human GSK3 nucleic acid and predicted amino acid sequences.

The term “glycogen synthase kinase 3” or “GSK3” as used herein refers to GSK3α or GSK3α. GSK3 is a protein originally identified by its phosphorylation of glycogen synthase as described in Woodgett et al, Trends Biochem Sci, 16: 177-181 (1991). Synonyms of GSK3 are tau protein kinase I (TPK I), FA kinase and kinase FA. Mammalian forms of GSK3 have been cloned as described in Woodgett, EMBO J. 9(8): 2431-2438 (1990), and He et al, Nature 374: 617-22 (1995) and Stambolic and Woodgett, Biochem. J. 303: 701-704 (1994). Modulators of GSK3 (GSK3 kinase inhibitors, GSK3 activators) may be modulators of any of the known forms of GSK3, including either GSK3α or GSK3β or both. GSK3 polypeptide as used herein includes the native protein and also may further include truncations, variants, alleles, analogs and derivatives of a native GSK3 protein. Such polypeptides possess one or more of the bioactivities of the GSK3 protein, including kinase activities such as polymerizing tau protein, or phosphorylating glycogen synthase, for example.

A “GSK3 nucleic acid”, as used herein, refers to a nucleic acid molecule which: (1) hybridizes under stringent conditions to a nucleic acid having the sequence of SEQ ID NOS: 1, 9, 11 or 13 and (2) codes for an GSK3 polypeptide. The preferred GSK3 nucleic acid molecule hydridizes under stringent conditions to the nucleic acid having the sequence of SEQ ID NO: 1 (nucleic acid sequence SEQ ID NO: 1 from U.S. Pat. No. 6,248,599 which is incorporated herein by reference). In its active form, the GSK3 polypeptide molecule inhibits an endothelial cell activity (e.g., endothelial cell viability, endothelial cell migration, angiogenesis). In its inactive form, the GSK3 polypeptide molecule enhances an endothelial cell activity, and in particular, inhibits apoptotic cell death of vascular endothelial cells. The preferred GSK3 nucleic acid molecule has the nucleic acid sequence of SEQ ID NO: 1, 9, 11, or 13. The GSK3 nucleic acids of the invention also include homologs and alleles of a nucleic acid molecule having the sequence of SEQ ID NOs. 1, 9, 11, or 13, as well as functionally equivalent fragments, variants, and analogs of the foregoing nucleic acids. “Functionally equivalent”, in reference to a GSK3 nucleic acid fragment, variant, or analog, refers to a nucleic acid that codes for a GSK3 polypeptide that, in its active form inhibits an endothelial cell activity and that in its inactive form, enhances an endothelial cell activity. Preferably the active GSK3 polypeptide molecule maintains a serine-threonine protein kinase activity. More specifically, “functionally equivalent” in reference to an active GSK3 polypeptide molecule refers to a GSK3 polypeptide molecule that has a serine-threonine protein kinase activity and is capable of inhibiting an endothelial cell activity. Conversely, “functionally equivalent” in reference to an inactive GSK3 polypeptide molecule refers to a GSK3 polypeptide molecule that has reduced or no serine-threonine protein kinase activity and is capable of enhancing an endothelial cell activity.

According to one aspect of the invention, a method for inhibiting angiogenesis is provided. The method involves administering to a subject in need of such treatment an angiogenesis inhibitor in an amount effective to inhibit angiogenesis in the subject. The angiogenesis inhibitor may be an “active GSK3 molecule” or a “GSK3 kinase activator”.

As used herein, an “active GSK3 molecule” refers to a GSK3 molecule that has a protein kinase activity, i.e., the GSK3 molecule has an enzymatic activity that permits it to phosphorylate a protein substrate. Preferably, the GSK3 molecule is a GSK3 nucleic acid molecule having SEQ ID NOS: 1, 9, 11, or 13, or is a GSK3 polypeptide molecule having SEQ ID NOS: 2, 3, 4, 5, 6, 7, 8, 10, 12, or 14. Thus, the GSK3 molecule as used herein, may be an active GSK3 nucleic acid molecule (i.e., a nucleic acid that encodes an active GSK3 polypeptide) or the encoded active GSK3 polypeptide molecule. Additionally, the active GSK3 molecule may be a nucleic acid or encoded polypeptide that is constitutively active, i.e., the sequence of the GSK3 nucleic acid molecule or of the GSK3 polypeptide molecule has been altered to prevent phosphorylation of the GSK3 polypeptide molecule (e.g., by an Akt molecule). (See, e.g., the Examples for an exemplary constitutively active GSK3 molecule.) Although not wishing to be bound to any particular theory or mechanism, it is believed that the naturally occurring active GSK3 molecule is not phosphorylated and that phosphorylation of the GSK3 molecule (e.g., by an upstream protein kinase such as an Akt molecule) inhibits GSK3 kinase activity. Akt molecules are described in U.S. Ser. No. 09/408,905, entitled: AKT COMPOSITIONS FOR ENHANCING SURVIVAL OF CELLS, filed Sep. 29, 1999, the entire contents of which are incorporated herein by reference. As noted in the cited application, Akt molecules include wild-type Akt molecules and constitutively-active Akt molecules.

Alternatively, the angiogenesis inhibitor may be a “GSK3 kinase activator”. As used herein, a “GSK3 kinase activator” refers to a molecule that is capable of mediating a transition from an inactive GSK3 molecule (e.g., a GSK3 molecule that has been phosphorylated by an Akt molecule) to an active GSK3 molecule having a protein kinase activity. GSK3 kinase activators may be identified in screening assays which identify agents which mediate the transition from an inactive to an active GSK3 molecule (e.g., by observing an inhibition of an endothelial cell activity such as viability, migration, angiogenesis in the presence of the putative GSK3 kinase activator). Screening methods and libraries containing candidate GSK3 kinase activators or inhibitors are described in detail below.

Subjects in need of inhibiting angiogenesis include subjects diagnosed as having a condition associated with undesirable endothelial cell proliferation (e.g., a cancer associated with excessive endothelial cell proliferation), or a predisposition to any of the foregoing conditions. The subject may or may not have a condition calling for treatment with an Akt inhibitor or an agent that downregulates expression of an AKT molecule in the subject. In some embodiments, the angiogenesis inhibitor is administered acutely to prevent future or further angiogenesis (e.g., to prevent further angiogenesis associated with a solid tumor). In some embodiments, acute administration of the angiogenesis inhibitor is to and/or in the area adjacent a solid tumor.

According to yet another aspect of the invention, a method for enhancing angiogenesis is provided. The method involves administering to a subject in need of such treatment an “angiogenesis promoter” in an amount effective to enhance angiogenesis in the subject. The angiogenesis promoter is an “inactive GSK3 molecule” or a “GSK3 kinase activity inhibitor”.

As used herein, an “inactive GSK3 molecule” refers to a GSK molecule which has reduced or no kinase activity compared to a wild-type GSK3 molecule (e.g., compared to a wild-type human GSK3 molecule such as SEQ ID NO: 1 or 2). Inactive GSK3 molecules include nucleic acid molecules and polypeptide molecules. In certain embodiments, the inactivated GSK3 molecules are GSK3 polypeptides that have been phosphorylated (e.g., by an Akt molecule).

As used herein, a GSK3 kinase inhibitor refers to a molecule which is capable of mediating a transition from an active GSK3 molecule which has a kinase activity to an inactive GSK3 molecule having no or reduced protein kinase activity. GSK3 kinase inhibitors may be identified in screening assays which identify agents which mediate the transition from an active to an inactive GSK3 molecule (e.g., by observing an enhancement of an endothelial cell activity such as viability, migration, or angiogenesis in the presence of the putative GSK3 kinase inhibitor). In certain embodiments, GSK3 kinase inhibitors exclude one or more known protein kinases (e.g., Akt) which phosphorylate and, thereby, inhibit GSK3 kinase activity.

Subjects in need of enhancing angiogenesis include subjects with myocardial infarction, ischemia-reperfusion injury, dilated cardiomyopathy, and conductive system disorders. In some embodiments, a growth factor may be co-administered. In some embodiments, Insulin-like Growth Factor-1 (IGF-1) is the growth factor utilized. In some embodiments, the angiogenesis promoter is administered acutely to prevent future or further tissue damage (e.g., cardiac tissue necrosis). In some embodiments, acute administration of the angiogenesis promoter is to the apical and anterolateral free wall of the heart. In these and or other embodiments, the subject may or may not have a condition calling for treatment with an AKT molecule or molecule that upregulates expression of an AKT molecule in the subject. In some embodiments, the subject does not have a condition calling for treatment with an angiogenesis promoter of the invention (i.e., an inactive GSK3 molecule and/or GSK3 kinase inhibitor have not been prescribed or administered to the subject for treatment or as part of a clinical trial).

In some embodiments, the invention involves co-administration of at least one anti- arteriosclerotic agent used in the treatment of an arteriosclerotic condition, with at least one angiogenesis promoter. In some embodiments, the anti-arteriosclerotic agent is selected from the group consisting of a HMG-CoA reductase inhibitor, a diuretic, an antiadrenergic agent, a vasodilator, a calcium channel antagonist, an angiotensin-converting enzyme (ACE) inhibitor, an angiotensin II antagonist and a clot dissolver together with an angiogenesis promoter to treat myocardial infarction and inhibit endothelial cell death (particularly, vascular endothelial cell death) in the subject.

The method of treatment according to this aspect of the invention is useful for treating myocardial infarction in a subject. “Myocardial infarction” is a focus of necrosis resulting from inadequate perfusion of the cardiac tissue. Myocardial infarction generally occurs with the abrupt decrease in coronary blood flow such as that follows a thrombotic occlusion of a coronary artery previously narrowed by atherosclerosis. Generally, infarction occurs when an atherosclerotic plaque fissures, ruptures, or ulcerates, and a mural thrombus forms leading to coronary artery occlusion.

The diagnosis of myocardial infarction in a subject determines the need for treating the subject according to the methods of the invention. A number of laboratory tests, well known in the art, are described, for example, in Harrison's Principles of Internal Medicine, 15^{th} Edition, (McGraw Hill, Inc., New York). Generally, the tests may be divided into four main categories: (1) nonspecific indexes of tissue necrosis and inflammation, (2) electrocardiograms, (3) serum enzyme changes (e.g., creatine phosphokinase levels), and (4) cardiac imaging. A person of ordinary skill in the art could easily apply any of the foregoing tests to determine when a subject is at risk, is suffering, or has suffered, a myocardial infarction. A positively identified subject would thus benefit from a method of treatment of the invention.

According to the invention, the method involves administering to a subject having a myocardial infarction an angiogenesis promoter selected from the group consisting of: (1) an inactive GSK3 molecule, and (2) a GSK3 kinase inhibitor, in an amount effective to inhibit cardiac tissue necrosis in the subject. By “having a myocardial infarction” it is meant that the subject is at risk of developing, is currently having, or has suffered a myocardial infarction. It is believed that immediate administration of an angiogenesis promoter (e.g., inactive GSK3 molecule) would greatly benefit the subject by inhibiting apoptotic cell death of endothelial cells prior to, or following the infarct. By “immediate” it is meant that administration occurs before (if it is diagnosed in time), or within 48 hours of the myocardial infarct, although administration up to 14 days after the episode may also be beneficial to the subject.

In some embodiments, when angiogenesis promoters such as inactive GSK3 molecules are used in the treatment of diseases associated with apoptotic endothelial cell death (e.g., myocardial infarction, ischemia-reperfusion injury, dilated cardiomyopathy, conductive system disorders and the like), a growth factor may be co-administered. In some embodiments, Insulin-like Growth Factor-1 (IGF-1) is the growth factor of choice. In some embodiments, constitutively inactive GSK3 molecules are utilized in the treatment of diseases associated with apoptotic endothelial cell-death, since their use negates the co-administration of a growth factor. In other words, no growth factor co-administration is necessary when the constitutively inactive form of the GSK3 molecule (e.g., a form which cannot be phosphorylated by Akt) is utilized.

The co-administered growth factor may act cooperatively, additively or synergistically with a wild-type GSK3 molecule of the invention to inhibit apoptotic cell death of endothelial cells, conferring to them enhanced viability. The growth factor is administered in effective amounts. Such amounts may be less than these sufficient to provide a therapeutic benefit when the growth factor is administered alone and not in combination with an angiogenesis promoter such as an inactive GSK3 molecule. A person of ordinary skill in the art would be able to determine the effective amounts needed (see description below).

Methods of administration for the GSK3 molecules of the invention (including inactive GSK3 molecules, active GSK3 molecules, GSK3 kinase activators, and GSK3 kinase inhibitors) in the treatment of the conditions identified herein include intraarterial administration. Intraarterial administration may be accompanied with a permeabilizing agent (e.g., nitric oxide), allowing easier access of the GSK3 molecules of the invention (and modulators of these molecules) into a preselected target location (e.g., the myocardium) via the circulation.

The angiogenesis promoters of the invention are particularly useful for inhibiting apoptotic cell death of vascular endothelial cells. The method involves administering to the subject an isolated inactive GSK3 molecule and/or a GSK3 kinase inhibitor in an amount and in a manner effective to inhibit apoptotic cell death of a vascular endothelial cell. Exemplary conditions that are caused by increased apoptotic cell death of a vascular endothelial cell are known to those of ordinary skill in the art and include, but are not limited to, vessel wall disease, and vascular endothelial cell dysfunction associated with hyperlipidemic subjects.

A “hyperlipidemic” subject is a hypercholesterolemic subject, a hypertriglyceridemic subject, or a hypercholesterolemic and hypertriglyceridemic subject. The current criteria for hyperlipidemia established for human subjects are well known in the art (See, e.g., Harrison's Principles of Internal Medicine, 15^{th} Edition, McGraw-Hill, Inc., New York). Hypercholesterolemic subjects and/or hypertriglyceridemic subjects are associated with increased incidence of premature coronary heart disease including vascular endothelial cell dysfunction. A hypercholesterolemic subject has an low-density lipoprotein (LDL) level of >160 mg/dL, or and LDL level of >130 mg/dL and at least two risk factors selected from the group consisting of male gender, family history of premature coronary heart disease, cigarette smoking (more than 10 per day), hypertension, high-density lipoprotein HDL (<35 mg/dL), diabetes mellitus, hyperinsulinemia, abdominal obesity, high lipoprotein (a), and personal history of cerebrovascular disease or occlusive peripheral vascular disease. A hypertriglyceridemic subject has a triglyceride (TG) level of >250 mg/dL. Thus, a hyperlipidemic subject is defined as one whose cholesterol and/or triglyceride levels equal or exceed the limits set as described above for either or both the hypercholesterolemic and hypertriglyceridemic subjects.

Methods of administration for the angiogenesis promoters of the invention into subjects with apoptotic cell death of vascular endothelial cells include intraarterial administration with clamping or locally via a balloon catheter (see later discussion). For example, in the case of intraarterial administration with clamping, the vessel wall in need of such treatment is “isolated” by clamping of the vessel on either side of the “injury” site, resulting in the temporary occlusion of the region to be treated, and allowing local delivery of the angiogenesis promoters (e.g., by injection). In the case of intraarterial administration via a balloon catheter, the catheter is of the “soft-hydrogel surface” type.

The term “to permit entry” of an angiogenesis promoter or of an angiogenesis inhibitor of the invention into a cell according to the invention has the following meanings depending upon the nature of the angiogenesis promoter or angiogenesis inhibitor. For a GSK3 nucleic acid molecule it is meant to describe entry of the nucleic acid through the cell membrane and into the cell nucleus, whereupon the “GSK3 transgene” can utilize the cell machinery to produce functional GSK3 polypeptide molecules. By “GSK3 transgene” it is meant to describe all of the GSK3 nucleic acid molecules of the invention, including the “wild-type GSK3” and the constitutively active GSK3 nucleic acid molecules with or without the associated vectors. For a GSK3 polypeptide molecule, the term “to permit entry” is meant to describe entry of the polypeptide through the cell membrane and into the cell cytoplasm, and utilization of the cell cytoplasmic machinery to produce a functional GSK3 polypeptide molecule (e.g., an active GSK3 polypeptide molecule that inhibits an endothelial cell function such as viability, migration, angiogenesis; or an inactive GSK3 polypeptide molecule that enhances any of the foregoing endothelial cell functions, and/or that inhibits apoptotic cell death of endothelial cells, and in particular, inhibits apoptotic cell death of vascular endothelial cells). Preferably the active GSK3 polypeptide maintains a serine-threonine protein kinase activity.

The angiogenesis inhibitors and angiogenesis promoters of the invention are administered in effective amounts. An effective amount is a dosage of the such molecules (e.g., a GSK3 nucleic acid molecule) sufficient to provide a desired result. The effective amount will vary with the nature of the drug, the particular condition being treated, the age and physical condition of the subject being treated, the severity of the condition, the duration of the treatment, the nature of the concurrent therapy (if any), the specific route of administration and like factors within the knowledge and expertise of the health practitioner. For example, in connection with apoptotic endothelial cell death during myocardial infarction, an effective amount is that amount which slows or inhibits the apoptotic endothelial cell death associated with myocardial infarction. Thus, it will be understood that the angiogenesis inhibitors and angiogenesis promoters of the invention may be used to treat the above-noted conditions prophylactically in subjects at risk of developing the foregoing conditions. By “acutely” it is meant that the angiogenesis inhibitors and angiogenesis promoters of the invention are administered immediately and according to the preferred modes of administration of the particular disorder being treated. For example, in connection with endothelial apoptotic cell death during myocardial infarction, the angiogenesis promoters will be administered to a subject in need of such treatment preferably by intra-coronary (and including cross-clamping of the aorta) or intra-myocardial injection (see e.g., Hajjar R J, et al., Proc Natl Acad Sci USA, 1998, 95:5251-6). As used in the claims, “inhibit” embraces preventing and/or reducing in all of the foregoing. It is preferred generally that a maximum dose be used, that is, the highest safe dose according to sound medical judgment.

A subject, as used herein, refers to any mammal (preferably a human, and including a non-human primate, cow, horse, pig, sheep, goat, dog, cat or rodent) that may be susceptible to a condition associated with apoptotic cell death of a cell (such as the conditions described above). Preferably the mammal is otherwise free of symptoms calling for Akt treatment and/or calling for treatment with an angiogenesis modulator of the invention. Reported conditions that have symptoms calling for treatment with a GSK3 molecule may also include conditions associated with apoptotic cell death of cell types other than endothelial cells which express GSK3 polypeptides.

The invention also contemplates methods for inhibiting apoptotic cell death in endothelial cells, particularly vascular endothelial cells. The method involves contacting an angiogenesis promoter (e.g., an inactive GSK3 molecule) with an endothelial cell in an amount effective to inhibit apoptotic cell death of the endothelial cell. In some embodiments the methods involves contacting an angiogenesis promoter (e.g. an inactive GSK3 molecule with an endothelial cell under conditions to permit entry of the angiogenesis promoter into the cell type of choice, in an amount effective to inhibit apoptotic cell death of the endothelial cell. In certain embodiments, the contacting of an angiogenesis promoter with an endothelial cell may comprise either acute or prophylactic administration of the angiogenesis promoter. Such acute and/or prophylactic administration of the angiogenesis promoter is particularly contemplated when the cell type of choice is part of a tissue or an organ scheduled to be transplanted or implanted. Administration of the angiogenesis promoters of the invention allows for longer term viability of the cells of the transplanted (implanted) tissue and/or organ under the adverse conditions the tissue and/or organ is subjected to during such procedure, i.e., ischemia, lower temperature, reperfusion, etc. therefore improving the tissue/organ's viability and/or acceptance by the recipient organism.

The same methods and modes of administration for the angiogenesis promoters of the invention may be used for the angiogenesis inhibitors of the invention, the difference being in the selection of subjects having a condition that may be treated by administration of these different molecules. For ease of discussion, angiogenesis promoters and angiogenesis inhibitors are collectively referred to herein as “angiogenesis modulators”. In general, angiogenesis promoters (inactive GSK3 molecules and GSK3 kinase inhibitors) are useful for treating conditions in which an enhanced endothelial cell activity (e.g., cell viability, migration, angiogenesis) is desirable. Conversely, angiogenesis inhibitors (active GSK3 molecules and GSK3 kinase activators) are useful for treating conditions in which inhibition of an endothelial cell activity is desirable. Despite these divergent applications, the same methods and modes of administration are useful for each of the foregoing categories of molecules.

When used therapeutically, the isolated angiogenic modulators of the invention are administered in therapeutically effective amounts. In general, a therapeutically effective amount means that amount necessary to delay the onset of, inhibit the progression of, or halt altogether the particular condition being treated. Generally, a therapeutically effective amount will vary with the subject's age, condition, and sex, as well as the nature and extent of the disease in the subject, all of which may be determined by one of ordinary skill in the art. The dosage may be adjusted by the individual physician or veterinarian, particularly in the event of any complication. A therapeutically effective amount typically varies from 0.01 mg/kg to about 1000 mg/kg, preferably from about 0.1 mg/kg to about 200 mg/kg, and most preferably from about 0.2 mg//kg to about 20 mg/kg, in one or more dose administrations daily, for one or more days.

The therapeutically effective amount of the isolated angiogenesis inhibitor is that amount effective to inhibit an endothelial cell activity particularly, in a vascular endothelial cell, and may be determined using, for example, standard tests known in the art.

The therapeutically effective amount of the isolated angiogenesis promoter is that amount effective to enhance an endothelial cell activity and, in particular, in a vascular endothelial cell, and also may be determined using, for example, standard tests known in the art. For example, TUNEL staining, and the appearance of condensed chromatin and other morphological features characteristic of apoptosis in electron micrographs may be used to assess apoptosis in the cells of the invention and other cell types.

Optionally, in one embodiment of the invention for treating myocardial infarction, an isolated angiogenesis promoter of the invention is administered to a subject in need of such treatment in combination with a method for treating an arteriosclerotic condition. An arteriosclerotic condition, as used herein, is a term of art that refers to classical atherosclerosis, accelerated atherosclerosis, atherosclerotic lesions and other physiological conditions characterized by undesirable vascular smooth muscle cell proliferation. See, e.g., Harrison's Principles of Internal Medicine (15^{th} Edition McGraw-Hill, Inc., New York) for a more detailed description of these conditions. The method for treating an arteriosclerotic condition may be a surgical method, an agent for treating restenosis, a method involving a drug therapy (e.g., gene therapy), or a combination of the foregoing.

Surgical methods for treating an arteriosclerotic condition include procedures such as bypass surgery, atherectomy, laser procedures, ultrasonic procedures, stenting, and balloon angioplasty. The angiogenesis promoters of the invention may be used to promote wound healing by inhibiting restenosis associated with balloon angioplasty. In some embodiments of the invention, the isolated angiogenesis promoter is administered to a subject in combination with a balloon angioplasty procedure. Alternatively or additionally, the angiogenesis promoter is administered systemically to a subject undergoing, about to undergo, or following balloon angioplasty. A balloon angioplasty procedure involves inserting a catheter having a deflated balloon into an artery. The deflated balloon is positioned in proximity to the atherosclerotic plaque and is inflated such that the plaque is compressed against the vascular wall. As a result, the balloon surface is in contact with a layer of vascular endothelial cells on the surface of the vessel. The isolated angiogenesis promoter molecule is attached to the balloon angioplasty catheter in a manner which permits release of the isolated angiogenesis promoter molecule at the site of the atherosclerotic plaque. The isolated angiogenesis promoter molecule may be attached to the balloon angioplasty catheter in accordance with standard procedures known in the art. For example, the isolated angiogenesis promoter molecule may be stored in a compartment of the balloon angioplasty catheter until the balloon is inflated, at which point it is released into the local environment. Alternatively, the isolated angiogenesis promoter molecule may be impregnated on the balloon surface, such that it contacts the cells of the arterial wall as the balloon is inflated. The angiogenesis promoter molecule also may be delivered in a perforated balloon catheter such as those disclosed in Flugelman, et al., Circulation, 85: 1110-1117(1992). See also, e.g., published PCT Patent Application WO 95/23161, for an exemplary procedure for attaching a therapeutic protein to a balloon angioplasty catheter. This procedure may be modified using no more that routine experimentation to attach a therapeutic nucleic acid or polypeptide to the balloon angioplasty catheter.

Additionally, the angiogenesis promoter molecule may be co-administered with an anti-arteriosclerotic agent for treating or preventing clinically significant arteriosclerosis. The term “co-administered” as used herein means administered substantially simultaneously with another agent. By substantially simultaneously, it is meant that an angiogenesis promoter of the invention (e.g., an inactive GSK3 molecule) is administered to the subject close enough in time with the administration of the other agent (e.g., an anti-arteriosclerotic agent, growth factor, etc.).

Anti-arteriosclerotic agents used in combination with the angiogenesis promoters of the invention include but are not limited to, the following drugs: HMG-CoA reductase inhibitors, diuretics, antiadrenergic agents, vasodilators, calcium channel antagonists, angiotensin-converting enzyme (ACE) inhibitors, angiotensin II antagonists, and clot dissolvers.

“HMG-CoA reductase (3-hydroxy-3-methylglutaryl-coenzyme A)” is the microsomal enzyme that catalyzes the rate-limiting reaction in cholesterol biosynthesis. An “HMG-CoA reductase inhibitor” inhibits HMG-CoA reductase, and therefore inhibits the synthesis of cholesterol. There is a large number of compounds described in the art that have been obtained naturally or synthetically, which have been seen to inhibit HMG-CoA reductase, and which form the category of agents useful for practicing the present invention. Traditionally these agents have been used to treat individuals with hypercholesterolemia. Examples include some which are commercially available, such as simvastatin (U.S. Pat. No. 4,444,784), lovastatin (U.S. Pat. No. 4,231,938), pravastatin sodium (U.S. Pat. No. 4,346,227), fluvastatin (U.S. Pat. No. 4,739,073), atorvastatin (U.S. Pat. No. 5,273,995), cerivastatin, and numerous others described in U.S. Pat. No. 5,622,985, U.S. Pat. No. 5,135,935, U.S. Pat. No. 5,356,896, U.S. Pat. No. 4,920,109, U.S. Pat. No. 5,286,895, U.S. Pat. No. 5,262,435, U.S. Pat. No. 5,260,332, U.S. Pat. No. 5,317,031, U.S. Pat. No. 5,283,256, U.S. Pat. No. 5,256,689, U.S. Pat. No. 5,182,298, U.S. Pat. No. 5,369,125, U.S. Pat. No. 5,302,604, U.S. Pat. No. 5,166,171, U.S. Pat. No. 5,202,327, U.S. Pat. No. 5,276,021, U.S. Pat. No. 5,196,440, U.S. Pat. No. 5,091,386, U.S. Pat. No. 5,091,378, U.S. Pat. No. 4,904,646, U.S. Pat. No. 5,385,932, U.S. Pat. No. 5,250,435, U.S. Pat. No. 5,132,312, U.S. Pat. No. 5,130,306, U.S. Pat. No. 5,116,870, U.S. Pat. No. 5,112,857, U.S. Pat. No. 5,102,911, U.S. Pat. No. 5,098,931, U.S. Pat. No. 5,081,136, U.S. Pat. No. 5,025,000, U.S. Pat. No. 5,021,453, U.S. Pat. No. 5,017,716, U.S. Pat. No. 5,001,144, U.S. Pat. No. 5,001,128, U.S. Pat. No. 4,997,837, U.S. Pat. No. 4,996,234, U.S. Pat. No. 4,994,494, U.S. Pat. No. 4,992,429, U.S. Pat. No. 4,970,231, U.S. Pat. No. 4,968,693, U.S. Pat. No. 4,963,538, U.S. Pat. No. 4,957,940, U.S. Pat. No. 4,950,675, U.S. Pat. No. 4,946,864, U.S. Pat. No. 4,946,860, U.S. Pat. No. 4,940,800, U.S. Pat. No. 4,940,727, U.S. Pat. No. 4,939,143, U.S. Pat. No. 4,929,620, U.S. Pat. No. 4,923,861, U.S. Pat. No. 4,906,657, U.S. Pat. No. 4,906,624 and U.S. Pat. No. 4,897,402, the disclosures of which patents are incorporated herein by reference.

Diuretics include but are not limited to thiazides, e.g., hydrochlorothiazide; loop diuretics, e.g., furosemide; and potassium-sparing diuretics, e.g., spironolactone, triamterene, and amiloride.

Antiadrenergic agents include but are not limited to clonidine; guanabenz; guanfacine; methyldopa; trimethapajn; Rauwolfia alkaloids, e.g., reserpine; guanethidine; guanadrel; phentolamine; phenoxybenzamine; prazosin; terazosin; propranolol; metoprolol; nadolol; atenolol; timolol; timdolol; acebutolol; and labetalol.

Vasodilators include but are not limited to hydralazine; minoxidil; diazoxide; and nitroprusside.

Calcium channel antagonists include but are not limited to nifedipine; diltiazem; and verapamil.

Angiotensin II antagonists are compounds which interfere with the activity of angiotensin 11 by binding to angiotensin 11 receptors and interfering with its activity. Angiotensin II antagonists are well known and include peptide compounds and non-peptide compounds. Most angiotensin II antagonists are slightly modified congeners in which agonist activity is attenuated by replacement of phenylalanine in position 8 with some other amino acid; stability may be enhanced by other replacements that slow degeneration in vivo. Examples of angiotensin 11 antagonists include but are not limited to: peptidic compounds (e.g., saralasin, [(San¹)(Val⁵)(Ala⁸)] angiotensin-(1-8) octapeptide and related analogs); N-substituted imidazole-2-one (U.S. Pat. No. 5,087,634); imidazole acetate derivatives including 2-N-butyl-4-chloro-1-(2-chlorobenzile) imidazole-5-acetic acid (see Long et al., J. Pharmacol. Exp. Ther. 247(1), 1-7 (1988)); 4, 5, 6, 7-tetrahydro-1H-imidazo [4, 5-c] pyridine-6-carboxylic acid and analog derivatives (U.S. Pat. No. 4,816,463); N2-tetrazole beta-glucuronide analogs (U.S. Pat. No. 5,085,992); substituted pyrroles, pyrazoles, and tryazoles (U.S. Pat. No. 5,081,127); phenol and heterocyclic derivatives such as 1, 3-imidazoles (U.S. Pat. No. 5,073,566); imidazo-fused 7-member ring heterocycles (U.S. Pat. No. 5,064,825); peptides (e.g., U.S. Pat. No. 4,772,684); antibodies to angiotensin II (e.g., U.S. Pat. No. 4,302,386); and aralkyl imidazole compounds such as biphenyl-methyl substituted imidazoles (e.g., EP Number 253,310, Jan. 20, 1988); ES8891 (N-morpholinoacetyl-(- I -naphthyl)-L-alanyl-(4, thiazolyl)-L-alanyl (35, 45)-4-amino-3-hydroxy-5-cyclo-hexapentanoyl-N-hexylamide, Sankyo Company, Ltd., Tokyo, Japan); SKF108566 (E-alpha-2-[2-butyl-1-(carboxy phenyl) methyl] 1H-imidazole-5-yl[methylane]-2-thiophenepropanoic acid, Smith Kline Beecham Pharmaceuticals, PA); Losartan (DUP753/MK954, DuPont Merck Pharmaceutical Company); Remikirin (RO42-5892, F. Hoffman LaRoche AG); A₂ agonists (Marion Merrill Dow) and certain non-peptide heterocycles (G. D.Searle and Company).

ACE, is an enzyme which catalyzes the conversion of angiotensin I to angiotensin II. ACE inhibitors include amino acids and derivatives thereof, peptides, including di- and tripeptides and antibodies to ACE which intervene in the renin-angiotensin system by inhibiting the activity of ACE, thereby reducing or eliminating the formation of pressor substance angiotensin II. ACE inhibitors have been used medically to treat hypertension, congestive heart failure, myocardial infarction and renal disease. Classes of compounds known to be useful as ACE inhibitors include but are not limited to acylmercapto and mercaptoalkanoyl prolines such as captopril (U.S. Pat. No. 4,105,776) and zofenopril (U.S. Pat. No. 4,316,906), carboxyalkyl dipeptides such as enalapril (U.S. Pat. No. 4,374,829), lisinopril (U.S. Pat. No. 4,374,829), quinapril (U.S. Pat. No. 4,344,949), ramipril (U.S. Pat. No. 4,587,258), and perindopril (U.S. Pat. No. 4,508,729), carboxyalkyl dipeptide mimics such as cilazapril (U.S. Pat. No. 4,512,924) and benazapril (U.S. Pat. No. 4,410,520), phosphinylalkanoyl prolines such as fosinopril (U.S. Pat. No. 4,337,201) and trandolopril.

Renin inhibitors are compounds which interfere with the activity of renin. Renin inhibitors include amino acids and derivatives thereof, peptides and derivatives thereof, and antibodies to renin. Examples of renin inhibitors that are the subject of United States patents are as follows: urea derivatives of peptides (U.S. Pat. No. 5,116,835); amino acids connected by nonpeptide bonds (U.S. Pat. No. 5,114,937); di- and tri- peptide derivatives (U.S. Pat. No. 5,106,835); amino acids and derivatives thereof (U.S. Pat. Nos. 5,104,869 and 5,095,119); diol sulfonamides and sulfinyls (U.S. Pat. No. 5,098,924); modified peptides (U.S. Pat. No. 5,095,006); peptidyl beta-aminoacyl aminodiol carbamates (U.S. Pat. No. 5,089,471); pyrolimidazolones (U.S. Pat. No. 5,075,451); fluorine and chlorine statine or statone containing peptides (U.S. Pat. No. 5,066,643); peptidyl amino diols (U.S. Pat. Nos. 5,063,208 and 4,845,079); N-morpholino derivatives (U.S. Pat. No. 5,055,466); pepstatin derivatives (U.S. Pat. No. 4,980,283); N-heterocyclic alcohols (U.S. Pat. No. 4,885,292); monoclonal antibodies to renin (U.S. Pat. No. 4,780,401); and a variety of other peptides and analogs thereof (U.S. Pat. Nos. 5,071,837, 5,064,965, 5,063,207, 5,036,054, 5,036,053, 5,034,512, and 4,894,437).

Drugs which are clot dissolvers include thrombolytic agents which have been used in the treatment of acute venous thromboembolism and pulmonary emboli and are well known in the art (e.g. see Hennekens et al., J Am Coll Cardiol, 25 (7 supp): 18S-22S (1995); Holmes, et al., J Am Coll Cardiol, 25 (7 suppl): IOS-17S(1995)). Thrombolytic agents include, for example, direct acting agents such as streptokinase and urokinase, and second generation agents such as tissue plasminogen activator (tPA).

Drugs which help contribute to the reduction of the plaque include cytostatic molecules and antisense agents to cell cycle regulatory molecules.

Certain cytokines function to strengthen the vascular wall by promoting endothelial cell proliferation. Cytokines which promote endothelial cell proliferation include, but are not limited to the following: vascular endothelial growth factor (VEGF), basic fibroblast growth factor (bFGF), and acidic fibroblast growth factor (aFGF). Substances that stimulate the proliferation or migration of normal endothelial cells include factors which are associated with the vascularization of tumors and substances which inhibit angiogenesis. Such substances include transforming growth factor beta (TGFβ), tumor necrosis factor alpha (TNFα), human platelet factor 4 (PF4), and alpha interferon (INF-α); factors which suppress cell migration, such as proteinase inhibitors, tissue inhibitors of metalloproteinase (TIMP-1 and TIMP-2); and other proteins such as protamine which has demonstrated angiostatic properties.

The above-described drug therapies are well known to those of ordinary skill in the art and are administered by modes known to those of skill in the art. The drug therapies are administered in amounts which are effective to achieve the physiological goals in combination with the isolated angiogenesis promoters of the invention.

An angiogenesis promoter may be administered alone or in combination with the above-described drug therapies as part of a pharmaceutical composition. Such a pharmaceutical composition may include the isolated angiogenesis promoter in combination with any standard physiologically and/or pharmaceutically acceptable carriers which are known in the art. The compositions should be sterile and contain a therapeutically effective amount of the isolated angiogenesis promoter in a unit of weight or volume suitable for administration to a patient. The term “pharmaceutically-acceptable carrier” as used herein means one or more compatible solid or liquid filler, diluents or encapsulating substances which are suitable for administration into a human or other animal. The term “carrier” denotes an organic or inorganic ingredient, natural or synthetic, with which the active ingredient is combined to facilitate the application. The components of the pharmaceutical compositions also are capable of being co-mingled with the molecules of the present invention, and with each other, in a manner such that there is no interaction which would substantially impair the desired pharmaceutical efficacy. Pharmaceutically acceptable further means a non-toxic material that is compatible with a biological system such as a cell, cell culture, tissue, or organism. The characteristics of the carrier will depend on the route of administration. Physiologically and pharmaceutically acceptable carriers include diluents, fillers, salts, buffers, stabilizers, solubilizers, and other materials which are well known in the art.

Although the invention is described above in reference to administering the angiogenesis promoters of the invention, it is to be understood that the angiogenesis inhibitors of the invention may be formulated and administered in a like manner as described herein in reference to the angiogenesis promoters.

Compositions suitable for parenteral administration conveniently comprise a sterile aqueous preparation of the angiogenesis promoters or angiogenesis inhibitors of the invention, which is preferably isotonic with the blood of the recipient. This aqueous preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed including synthetic mono- or di- glycerides. In addition, fatty acids such as oleic acid may be used in the preparation of injectables. Carrier formulations suitable for oral, subcutaneous, intravenous, intramuscular, etc. administrations may be found in Remington's Pharmaceutical Sciences, Mack Publishing Co., Easton, Pa.

A variety of administration routes are available. The particular mode selected will depend, of course, upon the particular drug selected, the severity of the condition being treated, and the dosage required for therapeutic efficacy. The methods of the invention, generally speaking, may be practiced using any mode of administration that is medically acceptable, meaning any mode that produces effective levels of the active compounds without causing clinically unacceptable adverse effects. Such modes of administration include oral, rectal, topical, nasal, interdermal, or parenteral routes. The term “parenteral” includes subcutaneous, intravenous, intramuscular, or infusion. Intravenous or intramuscular routes are not particularly suitable for long-term therapy and prophylaxis. They could, however, be preferred in emergency situations. Intramyocardial administration may be used in patients suffering from myocardial infarction. Oral administration will be preferred for prophylactic treatment because of the convenience to the patient as well as the dosing schedule.

The pharmaceutical compositions may conveniently be presented in unit dosage form and may be prepared by any of the methods well-known in the art of pharmacy. All methods include the step of bringing the angiogenesis promoters or angiogenesis inhibitors of the invention into association with a carrier which constitutes one or more accessory ingredients. In general, the compositions are prepared by uniformly and intimately bringing the angiogenesis promoters or angiogenesis inhibitors into association with a liquid carrier, a finely divided solid carrier, or both, and then, if necessary, shaping the product.

Compositions suitable for oral administration may be presented as discrete units, such as capsules, tablets, lozenges, each containing a predetermined amount of the angiogenesis promoters or angiogenesis inhibitors. Other compositions include suspensions in aqueous liquids or non-aqueous liquids such as a syrup, elixir or an emulsion.

Other delivery systems may include time-release, delayed release or sustained release delivery systems. Such systems may avoid repeated administrations of the angiogenesis promoters or angiogenesis inhibitors described above, increasing convenience to the subject and the physician. Many types of release delivery systems are available and known to those of ordinary skill in the art. They include the above-described polymeric systems, as well as polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono- di- and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which the angiogenesis promoter or angiogenesis inhibitor is contained in a form within a matrix such as those described in U.S. Pat. Nos. 4,452,775, 4,675,189, and 5,736,152, and (b) diffusional systems in which an active component permeates at a controlled rate from a polymer such as described in U.S. Pat. Nos. 3,854,480, 5,133,974 and 5,407,686. In addition, pump-based hardware delivery systems may be used, some of which are adapted for implantation.

Use of a long-term sustained release implant may be particularly suitable for treatment of chronic conditions. Long-term release, are used herein, means that the implant is constructed and arranged to deliver therapeutic levels of the active ingredient for at least 30 days, and preferably 60 days. Long-term sustained release implants are well-known to those of ordinary skill in the art and include some of the release systems described above.

The isolated angiogenesis modulators of the invention may be administered alone or in combination with the above-described drug therapies by any conventional route, including injection or by gradual infusion over time. The administration may, for example, be oral, intravenous, intraperitoneal, intramuscular, intra-cavity, subcutaneous, or transdermal. When using the isolated angiogenesis promoters of the invention, direct administration to the site with the increased apoptotic cell death of an endothelial cell (e.g., a vascular endothelial cell) such as administration by injection, is preferred (see also earlier description).

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

In general, the GSK3 nucleic acid molecules of the invention (active or inactive forms) may be administered to the subject (any mammalian recipient) using the same modes of administration that currently are used for gene therapy in humans (e.g., adenovirus-mediated gene therapy). In some embodiments, the GSK3 nucleic acid molecule (contained in, or associated with, an appropriate vector) is administered to the mammalian recipient by intra-vascular or intra-muscular injection. A procedure for performing in vivo gene therapy for delivering a nucleic acid for performing ex vivo gene therapy is outlined in U.S. Pat. No. 5,399,346. In general, ex vivo gene therapy involves introduction in vitro of a functional copy- of a gene or fragment thereof into a cell(s) of a subject and returning the genetically engineered cell(s) to the subject. The functional copy of the gene or fragment thereof is under operable control of regulatory elements which permit expression of the gene in the genetically engineered cell(s). Accordingly, the GSK3 nucleic acid molecules of the invention may be delivered to the cells of the invention, ex vivo or in vivo, e.g., by administering an inactive GSK3 nucleic acid molecule to treat excessive apoptotic cell death. Numerous transfection and transduction techniques as well as appropriate expression vectors are well known to those of ordinary skill in the art, some of which are described in PCT application WO95/00654.

As an illustrative example, a vector containing an inactive GSK3 nucleic acid molecule is delivered to a site of increased apoptotic cell death in a subject who is a candidate for such gene therapy. Then, the vector genetically modifies the cell in vivo with DNA (RNA) encoding an inactive GSK3 polypeptide molecule of the invention. Such genetically modified cells are expected to undergo apoptotic cell death at a reduced rate and their viability in vivo is enhanced.

In related aspects of the invention, a composition including an isolated active GSK3 nucleic acid molecule or a composition including an isolated inactive GSK3 nucleic acid molecule is provided. The isolated GSK3 nucleic acid molecule or the isolated inactive GSK3 molecule is operably linked to a gene expression sequence which permits expression of the active GSK3 nucleic acid molecule or of the inactive GSK3 nucleic acid molecule in an endothelial cell (e.g., vascular endothelial cell). In some preferred embodiments, the nucleic acid is contained in an appropriate expression vector (e.g., adenoviral vector, modified adenoviral vector, retroviral vector, plasmid, liposome) to more efficiently genetically modify the targeted cell and achieve expression of active GSK3 molecule or inactive GSK3 molecule in the targeted cell. In some embodiments, the vector is an adenoviral vector.

The term “isolated”, as used herein in reference to a nucleic acid molecule, means a nucleic acid sequence: (i) amplified in vitro by, for example, polymerase chain reaction (PCR); (ii) synthesized by, for example, chemical synthesis; (iii) recombinantly produced by cloning; or (iv) purified, as by cleavage and gel separation. The term “isolated”, as used herein in reference to a polypeptide (protein), means a polypeptide encoded by an isolated nucleic acid sequence, as well as polypeptides synthesized by, for example, chemical synthetic methods, and polypeptides separated from biological materials, and then purified using conventional protein analytical procedures.

In one embodiment, the GSK3 nucleic acid molecule has the nucleotide sequence of SEQ ID NO: 1 (“GSK3 wild-type nucleic acid”), the nucleotide sequence encoding a “wild-type GSK3 polypeptide”, i.e., the complete coding sequence of the gene encoding a human GSK3 polypeptide molecule.

In some embodiments of the methods, the GSK3 nucleic acid is selected from the group consisting of a wild-type GSK3 nucleic acid (e.g., SEQ ID NOS. 1, 9, 11, or 13), and a GSK3 nucleic acid which has been modified to encode a GSK3 polypeptide molecule that is constitutively active (see, e.g., the Examples). Constitutively active GSK3 molecules have an altered sequence which does not permit the GSK3 polypeptide molecule to be phosphorylated by an Akt molecule or other upstream protein kinase that phosphorylates GSK3 and, thereby inhibits its kinase activity.

The GSK3 nucleic acid molecule is operatively coupled to a promoter that can express GSK3 in a targeted cell (e.g., an endothelial cell such as a vascular endothelial cell). in some embodiments, the nucleic acid is contained in an appropriate expression vector (e.g., adenoviral vector, modified adenoviral vector, retroviral vector, plasmid, liposome) to more efficiently genetically modify the targeted cell and achieve expression of multiple copies of the GSK3 polypeptide molecule.

The GSK3 nucleic acid molecule, in one embodiment, is operably linked to a gene expression sequence which directs the expression of the GSK3 nucleic acid molecule within an endothelial cell. The “gene expression sequence” is any regulatory nucleotide sequence, such as a promoter sequence or promoter-enhancer combination, which facilitates the efficient transcription and translation of the GSK3 nucleic acid molecule to which it is operably linked. The gene expression sequence may, for example, be a mammalian or viral promoter, such as a constitutive or inducible promoter. Constitutive mammalian promoters include, but are not limited to, the promoters for the following genes: hypoxanthine phosphoribosyl transferase (HPRT), adenosine deaminase, pyruvate kinase, α-actin promoter and other constitutive promoters. Exemplary viral promoters which function constitutively in eukaryotic cells include, for example, promoters from the simian virus, papilloma virus, adenovirus, human immunodeficiency virus (HIV), Rous sarcoma virus, cytomegalovirus, the long terminal repeats (LTR) of moloney leukemia virus and other retroviruses, and the thymidine kinase promoter of herpes simplex virus. Other constitutive promoters are known to those of ordinary skill in the art. The promoters useful as gene expression sequences of the invention also include inducible promoters. Inducible promoters are expressed in the presence of an inducing agent. For example, the metallothionein promoter is induced to promote transcription and translation in the presence of certain metal ions. Other inducible promoters are known to those of ordinary skill in the art.

In general, the gene expression sequence shall include, as necessary, 5′ non-transcribing and 5′ non-translating sequences involved with the initiation of transcription and translation, respectively, such as a TATA box, capping sequence, CAAT sequence, and the like. Especially, such 5′ non-transcribing sequences will include a promoter region which includes a promoter sequence for transcriptional control of the operably joined GSK3 nucleic acid molecule. The gene expression sequences optionally includes enhancer sequences or upstream activator sequences as desired.

In some embodiments the GSK3 nucleic acid molecule of the invention is linked to a gene expression sequence which permits expression of the GSK3 nucleic acid molecule in an endothelial cell, particularly in a vascular endothelial cell. In some embodiments, the gene expression sequence permits expression of the GSK3 nucleic acid molecule in an endothelial cell and does not permit expression of the GSK3 nucleic acid molecule in other cell types. A sequence which permits expression of the GSK3 nucleic acid molecule in a cell such as a vascular endothelial cell, is one which is selectively active in such a cell type, thereby causing expression of the GSK3 nucleic acid molecule in these cells. The von Willebrand factor gene promoter, for example, may be used to express the GSK3 nucleic acid molecule in a vascular endothelial cell. Those of ordinary skill in the art will be able to easily identify alternative promoters that are capable of expressing a GSK3 nucleic acid molecule in the preferred endothelial cells of the invention.

The GSK3 nucleic acid sequence and the gene expression sequence are said to be “operably linked” when they are covalently linked in such a way as to place the transcription and/or translation of the GSK3 coding sequence under the influence or control of the gene expression sequence. If it is desired that the GSK3 sequence be translated into a functional protein, two DNA sequences are said to be operably linked if induction of a promoter in the 5′ gene expression sequence results in the transcription of the GSK3 sequence and if the nature of the linkage between the two DNA sequences does not (1) result in the introduction of a frame-shift mutation, (2) interfere with the ability of the promoter region to direct the transcription of the GSK3 sequence, or (3) interfere with the ability of the corresponding RNA transcript to be translated into a protein. Thus, a gene expression sequence would be operably linked to a GSK3 nucleic acid sequence if the gene expression sequence were capable of effecting transcription of that GSK3 nucleic acid sequence such that the resulting transcript might be translated into the desired protein or polypeptide.

The GSK3 nucleic acid molecules of the invention may be delivered to the preferred cell types of the invention alone or in association with a vector. In its broadest sense, a “vector” is any vehicle capable of facilitating: (1) delivery of a GSK3 molecule to a target cell and/or (2) uptake of a GSK3 molecule by a target cell. Preferably, the vectors transport the GSK3 molecule into the target cell with reduced degradation relative to the extent of degradation that would result in the absence of the vector. Optionally, a “targeting ligand” may be attached to the vector to selectively deliver the vector to a cell which expresses on its surface the cognate receptor for the targeting ligand. In this manner, the vector (containing a GSK3 nucleic acid molecule or a GSK3 polypeptide molecule) may be selectively delivered to an endothelial cell. Methodologies for targeting include conjugates, such as those described in U.S. Pat. No. 5,391,723 to Priest. Another example of a well-known targeting vehicle is a liposome. Liposomes are commercially available from Gibco BRL (Carlsbad, Calif.). Numerous methods are published for making targeted liposomes. Preferably, the GSK3 molecules of the invention are targeted for delivery to vascular endothelial cells.

In general, the vectors useful in the invention include, but are not limited to, plasmids, phagemids, viruses, other vehicles derived from viral or bacterial sources that have been manipulated by the insertion or incorporation of the nucleic acid sequences of the invention, and additional nucleic acid fragments (e.g., enhancers, promoters) which may be attached to the nucleic acid sequences of the invention. Viral vectors are preferred type of vector in some embodiments and include, but are not limited to, nucleic acid sequences from the following viruses: adenovirus; adeno-associated virus; retrovirus, such as moloney murine leukemia virus; harvey murine sarcoma virus; murine mammary tumor virus; rouse sarcoma virus; SV40-type viruses; polyoma viruses; Epstein-Barr viruses; papilloma viruses; herpes virus; vaccinia virus; polio virus; and RNA virus such as a retrovirus. One may readily employ other vectors not named but known in the art.

A preferred virus for certain applications is the adeno-associated virus, a double-stranded DNA virus. The adeno-associated virus is capable of infecting a wide range of cell types and species and may be engineered to be replication-deficient. It further has advantages, such as heat and lipid solvent stability, high transduction frequencies in cells of diverse lineages, including hemopoietic cells, and lack of superinfection inhibition thus allowing multiple series of transductions. Reportedly, the adeno-associated virus may integrate into human cellular DNA in a site-specific manner, thereby minimizing the possibility of insertional mutagenesis and variability of inserted gene expression. In addition, wild-type adeno-associated virus infections have been followed in tissue culture for greater than 100 passages in the absence of selective pressure, implying that the adeno-associated virus genomic integration is a relatively stable event. The adeno-associated virus may also function in an extrachromosomal fashion. Various preparations of vectors containing GSK3 nucleic molecules are provided in the Examples. See also, U.S. Pat. No. 6,248,559, which reports sequence information for GSK3β and related cloning methods, the entire contents of which patent are incorporated herein by reference.

In general, other useful viral vectors are based on non-cytopathic eukaryotic viruses in which non-essential genes have been replaced with the gene of interest. Non-cytopathic viruses include retroviruses, the life cycle of which involves reverse transcription of genomic viral RNA into DNA with subsequent proviral integration into host cellular DNA. Adenoviruses and retroviruses have been approved for human gene therapy trials. In general, the retroviruses are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell line with plasmid, production of recombinant retroviruses by the packaging cell line, collection of viral particles from tissue culture media, and infection of the target cells with viral particles) are provided in Kriegler, M., “Gene Transfer and Expression, A Laboratory Manual,” W. H. Freeman Co., New York (1990) and Murry, E. J. Ed. “Methods in Molecular Biology,” vol. 7, Humana Press, Inc., Cliffton, N.J. (1991).

Another retroviral vector is the vector derived from the moloney murine leukemia virus, as described in Nabel, E. G., et al., Science, 1990, 249:1285-1288. These vectors reportedly were effective for the delivery of genes to all three layers of the arterial wall, including the media. Other vectors are disclosed in Flugelman, et al., Circulation, 1992, 85:1110-1117. Additional vectors that are useful are described in U.S. Pat. No. 5,674,722 by Mulligan, et al.

In addition to the foregoing vectors, other delivery methods may be used to deliver a GSK3 molecule to an endothelial cell, and facilitate uptake thereby. These additional delivery methods include, but are not limited to, natural or synthetic molecules, other than those derived from bacteriological or viral sources, capable of delivering the isolated GSK3 molecule to a cell.

One such delivery method of the invention is a colloidal dispersion system. Colloidal dispersion systems include lipid-based systems including oil-in-water emulsions, micelles, mixed micelles, and liposomes. One colloidal system of the invention is a liposome. Liposomes are artificial membrane vessels which are useful as a delivery vector in vivo or in vitro. It has been shown that large unilamellar vesicles (LUV), which range in size from 0.2-4.0 μm may encapsulate large macromolecules. RNA, DNA, and intact virions may be encapsulated within the aqueous interior and be delivered to cells in a biologically active form (Fraley, et al., Trends Biochem. Sci., 1981, 6:77). In order for a liposome to be an efficient gene transfer vector, one or more of the following characteristics should be present: (1) encapsulation of the gene of interest at high efficiency with retention of biological activity; (2) preferential and substantial binding to a target cell in comparison to non-target cells; (3) delivery of the aqueous contents of the vesicle to the target cell cytoplasm at high efficiency; and (4) accurate and effective expression of genetic information.

Liposomes may be targeted to a particular tissue, such as the vascular cell wall, by coupling the liposome to a specific ligand such as a monoclonal antibody, sugar, glycolipid, or protein. Ligands which may be useful for targeting a liposome to the vascular wall include, but are not limited to the viral coat protein of the Hemagglutinating virus of Japan. Additionally, the vector may be coupled to a nuclear targeting peptide, which will direct the GSK3 nucleic acid to the nucleus of the host cell.

Liposomes are commercially available from Gibco BRL, for example, as LIPOFECTIN™ and LIPOFECTACE™, which are formed of cationic lipids such as N-[-(1-(2, 3 dioleyloxy)-propyl]-N, N, N-trimethylammonium chloride (DOTMA) and dimethyl dioctadecylammonium bromide (DDAB). Methods for making liposomes are well known in the art and have been described in many publications. Liposomes also have been reviewed by Gregoriadis, G. in Trends in Biotechnology, 3: 235-241 (1985). Novel liposomes for the intracellular delivery of macromolecules, including nucleic acids, are also described in PCT International application no. PCT/US96/07572 (Publication No. WO 96/40060, entitled “Intracellular Delivery of Macromolecules”).

In one particular embodiment, the vehicle is a biocompatible microparticle or implant that is suitable for implantation into the mammalian recipient. Exemplary bioerodible implants that are useful in accordance with this method are described in PCT International application no. PCT/US/03307 (Publication No. WO 95/24929, entitled “Polymeric Gene Delivery System”. PCT/US/03307 describes a biocompatible, preferably biodegradable polymeric matrix for containing an exogenous gene under the control of an appropriate promoter. The polymeric matrix is used to achieve sustained release of the exogenous gene in the patient. In accordance with the instant invention, the GSK3 nucleic acids described herein are encapsulated or dispersed within the biocompatible, preferably biodegradable polymeric matrix disclosed in PCT/US/03307. The polymeric matrix in some embodiments is in the form of a microparticle such as a microsphere (wherein the GSK3 nucleic acid is dispersed throughout a solid polymeric matrix) or a microcapsule (wherein the GSK3 nucleic acid is stored in the core of a polymeric shell). Other forms of the polymeric matrix for containing the GSK3 nucleic acid include films, coatings, gels, implants, and stents. The size and composition of the polymeric matrix device is selected to result in favorable release kinetics in the tissue into which the matrix device is implanted. The size of the polymeric matrix devise further is selected according to the method of delivery which is to be used, typically injection into a tissue or administration of a suspension by aerosol into the nasal and/or pulmonary areas. The polymeric matrix composition may be selected to have both favorable degradation rates and also to be formed of a material which is bioadhesive, to further increase the effectiveness of transfer when the devise is administered to a vascular surface. The matrix composition also may be selected not to degrade, but rather, to release by diffusion over an extended period of time.

Both non-biodegradable and biodegradable polymeric matrices may be used to deliver the GSK3 nucleic acids of the invention to the subject. Biodegradable matrices are preferred. Such polymers may be natural or synthetic polymers. Synthetic polymers are preferred in some embodiments. The polymer is selected based on the period of time over which release is desired, generally in the order of a few hours to a year or longer. Typically, release over a period ranging from between a few hours and three to twelve months is most desirable. The polymer optionally is in the form of a hydrogel that may absorb up to about 90% of its weight in water and further, optionally is cross-linked with multivalent ions or other polymers.

In general, the GSK3 nucleic acids of the invention are delivered using the bioerodible implant by way of diffusion, or more preferably, by degradation of the polymeric matrix. Exemplary synthetic polymers which may be used to form the biodegradable delivery system include: polyamides, polycarbonates, polyalkylenes, polyalkylene glycols, polyalkylene oxides, polyalkylene terepthalates, polyvinyl alcohols, polyvinyl ethers, polyvinyl esters, poly-vinyl halides, polyvinylpyrrolidone, polyglycolides, polysiloxanes, polyurethanes and co-polymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, polyvinyl chloride, polystyrene and polyvinylpyrrolidone.

Examples of non-biodegradable polymers include ethylene vinyl acetate, poly(meth) acrylic acid, polyamides, copolymers and mixtures thereof.

Examples of biodegradable polymers include synthetic polymers such as polymers of lactic acid and glycolic acid, polyanhydrides, poly(ortho)esters, polyurethanes, poly(butic acid), poly(valeric acid), and poly(lactide-cocaprolactone), and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion.

Bioadhesive polymers of particular interest include bioerodible hydrogels described by H. S. Sawhney, C. P. Pathak and J. A. Hubell in Macromolecules, 1993, 26, 581-587, the teachings of which are incorporated herein, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate). Thus, the invention provides a composition of the above-described GSK3 molecules for use as a medicament, methods for preparing the medicament and methods for the sustained release of the medicament in vivo.

Compaction agents may also be used in combination with a vector of the invention. A “compaction agent”, as used herein, refers to an agent, such as a histone, that neutralizes the negative charges on the nucleic acid and thereby permits compaction of the nucleic acid into a fine granule. Compaction of the nucleic acid facilitates the uptake of the nucleic acid by the target cell. The compaction agents may be used alone, i.e., to deliver the isolated GSK3 nucleic acid in a form that is more efficiently taken up by the cell or, more preferably, in combination with one or more of the above-described vectors.

Other exemplary compositions that may be used to facilitate uptake by a target cell of the GSK3 nucleic acid molecules include calcium phosphate and other chemical mediators of intracellular transport, microinjection compositions, electroporation and homologous recombination compositions (e.g., for integrating a GSK3 nucleic acid molecule into a preselected location within the target cell chromosome).

The GSK3 nucleic acid molecules code for a GSK3 polypeptide molecule. As used herein, a “GSK3 polypeptide” refers to a polypeptide that is coded for by a GSK3 nucleic acid molecule and/or a structurally related molecule, and preferably has serine-threonine protein kinase activity. GSK3 polypeptide molecules that are in their inactive form are useful for inhibiting apoptotic cell death of an endothelial cell and for enhancing an endothelial cell activity (e.g., viability, migration, angiogenesis). GSK3 polypeptide molecules that are in their active form are useful for inhibiting an endothelial cell activity (e.g., viability, migration, angiogenesis). The preferred GSK3 polypeptide molecule of the invention has the amino acid sequence of SEQ ID NOS. 2, 3, 4, 5, 6, 7, 8, 10, 12, or 14, or a functionally equivalent fragment of the foregoing sequences. More preferably, the GSK3 polypeptide molecule of the invention has the amino acid sequence of SEQ ID NO. 2. GSK3 polypeptide molecules further include functionally equivalent variants, and analogs of the foregoing sequences, provided that the fragments, variants, and analogs preferably maintain a serine-threonine kinase protein activity when in an active form, are capable of inhibiting apoptotic cell death of an endothelial cell when in an inactive form, are capable of enhancing an endothelial cell activity when in an inactive form, and/or are capable of inhibiting an endothelial cell activity when in an active form. The invention also embraces proteins and peptides coded for by any of the foregoing GSK3 nucleic acid molecules.

“Structurally related,” as used herein, refers to nucleic acids and polypeptides that are homologous and/or allelic to a GSK3 molecule. In general homologs and alleles typically will share at least 40% nucleotide identity and/or at least 50% amino acid identity to SEQ ID NOS:1, 9, 11, or 13, and SEQ ID NOS:2, 3, 4, 5, 6, 7, 8, 10, 12, or 13, respectively, in some instances will share at least 50% nucleotide identity and/or at least 65% amino acid identity and in still other instances will share at least 60% nucleotide identity and/or at least 75% amino acid identity. The homology can be calculated using various, publicly available software tools developed by NCBI (Bethesda, Md.) that may be obtained through the internet (ftp:/ncbi.nlm.nih.gov/pub/). Exemplary tools include the BLAST system available at http://wwww.ncbi.nlm.nih.gov. Pairwise and ClustalW alignments (BLOSUM30 matrix setting) as well as Kyte-Doolittle hydropathic analysis can be obtained using the MacVetor sequence analysis software (Oxford Molecular Group). Watson-Crick complements of the foregoing nucleic acids also are embraced by the invention.

The preferred GSK3 nucleic acid molecules of the invention encode the GSK3 polypeptide molecule having the amino acid sequence of SEQ ID NO. 2, the complete polypeptide sequence of the gene encoding the human GSK3P.

It will be appreciated by those skilled in the art that various modifications of the GSK3 polypeptide having the sequence of SEQ ID NOS: 2, 3, 4, 5, 6, 7, 8, 10, 12, or 14, or functionally equivalent fragments of the foregoing sequences, may be made without departing from the essential nature of the invention. Accordingly, it is intended that polypeptides which have the amino acid sequence of SEQ ID NOS: 2, 3, 4, 5, 6, 7, 8, 10, 12, or 14 but which include conservative substitutions are embraced within the instant invention. As used herein, “conservative amino acid substitution” refers to an amino acid substitution which does not alter the relative charge or size characteristics of the polypeptide in which the amino acid substitution is made. Conservative substitutions of amino acids include substitutions made amongst amino acids with the following groups: (1) M,I,L,V; (2) F,Y,W; (3) K,R,H; (4) A,G; (5) S,T; (6) Q,N; and, (7) E,D. Additionally, negatively charged amino acids (e.g., aspartic acid, glutamic acid) may be used in place of an amino acid that is phosphorylated to provide a phosphomimetic polypeptide which mimics the activity of a phosphorylated GSK3 polypeptide. Fusion proteins, in which a peptide of the invention is coupled to a solid support (such as a polymeric bead), a carrier molecule (such as keyhole limpet hemocyanin), or a reporter group (such as radiolabel or other tag), or a membrane anchoring group (such a myristoylation peptide) also are embraced within the invention.

According to yet another aspect of the invention, a method of screening for a GSK3 kinase modulator (activator or inhibitor) that modulates (enhances or inhibits) an endothelial cell activity is provided. The method involves: (a) contacting a test molecule with an endothelial cell under conditions to permit entry of the test molecule into the cell; and (b) determining whether the test molecule modulates an endothelial cell activity (e.g., viability, migration, angiogenesis). An increase in an endothelial cell activity in the presence of the test molecule indicates that the test molecule is a GSK3 kinase inhibitor; a decrease in an endothelial cell activity in the presence of the test molecule indicates that the test molecule is a GSK3 kinase activator. Test molecules may be members of a library of molecules such as a phage display library or a chemical combinatorial library. The screening method may be performed in vitro or in vivo (e.g., an animal model). (See also U.S. Pat. Nos. 6,057,117 and 6,057,118 which report screening methods and libraries for identifying agents that inhibit GSK3.)

Candidate GSK3 kinase modulators include small molecules that bind to the GSK3 polypeptide molecule and upregulate (e.g., positive allosteric effectors) or downregulate (e.g., negative allosteric effectors or competitive inhibitors) the activity of a GSK3 molecule with respect to modulating an endothelial cell activity. Exemplary small molecules include ATP analogs, which may be designed and selected to function as competitive, noncompetitive or suicide inhibitors. GSK3 kinase inhibitors also include peptides that mimic the natural substrate of GSK3 and, thereby, inhibit GSK3 kinase activity. The term “GSK3 substrate” refers to a peptide or a polypeptide or a synthetic peptide derivative that may be phosphorylated by GSK3 activity in the presence of an appropriate amount of ATP or a phosphate donor. Detection of the phosphorylated substrate is generally accomplished by the addition of a labeled phosphate that may be detected by some means common in the art of labeling, such as radiolabeled phosphate. The peptide substrate may be a peptide that resides in a molecule as a part of a larger polypeptide, or may be an isolated peptide designed for phosphorylation by GSK3. Exemplary GSK3 kinase activators include small molecules and peptides that bind to the regulatory domain of GSK3 and, thereby, induces or maintains the GSK3 polypeptide molecule in an active conformation.

Candidate GSK3 kinase modulators may be derived from almost any source of chemical libraries, naturally occurring compounds, or mixtures of compounds. Described below are some exemplary and possible sources of candidate GSK3 kinase modulators, synthesis of libraries of peptides, peptoids, and small organic molecules. The candidate GSK3 kinase modulators may also be polynucleotides, for example ribozymes or antisense molecules designed based on knowledge of GSK3 polynucleotide sequence.

The term “inhibitor”, as used in reference to a GSK3 kinase activity, refers to any inhibitor or antagonist of GSK3 activity. The GSK3 kinase inhibitor may be a peptide GSK3 antagonist, a peptoid GSK3 antagonist, a small organic molecule GSK3 antagonist or a polynucleotide GSK3 antagonist. It is expected that some inhibitors will act at transcription, some at translation, and some on the mature protein, for example, at the specific site of GSK3 that acts to phosphorylate another protein. However, the use and appropriateness of such inhibitors of GSK3 for the purposes of the invention are not limited to any theories of mechanism of action of the inhibitor. It is sufficient for purposes of the invention that an inhibitor inhibit the activity of GSK3, for example, and most particularly, the kinase activity of GSK3. This may be determined, for example, by observing enhancement of an endothelial cell activity in a screening assay performed in accordance with the methods of the invention. Exemplary inhibitors of GSK3 kinase activity are described in U.S. Pat. Nos. 6,057,117 and 6,153,618, the entire contents of which are incorporated herein by reference.

Conversely, the term “activator”, as used in reference to a GSK3 kinase activity, refers to any activator or agonist of GSK3 activity. The GSK3 kinase activator may be a peptide agonist (e.g., an allosteric peptide that binds to GSK3 and induces or maintains the GSK3 protein in an active conformation), a peptoid GSK3 agonist, a small organic molecule GSK3 agonist or a polynucleotide GSK3 agonist. It is expected that some agonists will act at transcription, some at translation, and some on the mature protein, for example, at the specific site of GSK3 that acts to induce or maintain an active confirmation. However, the use and appropriateness of such activators of GSK3 for the purposes of the invention are not limited to any theories of mechanism of action of the inhibitor. It is sufficient for purposes of the invention that an activator activate the activity of GSK3, for example, and most particularly, the kinase activity of GSK3. This may be determined, for example, by observing inhibition of an endothelial cell activity in a screening assay performed in accordance with the methods of the invention.

Analogs of peptides as used herein include peptides having one or more peptide mimics, for example peptoids that possess protein-like activity. Included within the definition are, for example, peptides containing one or more analogs of an amino acid (including, for example, unnatural amino acids), peptides with substituted linkages, as well as other modifications known in the art, both naturally occurring and not naturally occurring.

The term “small molecule” includes any chemical or other moiety that may act to affect biological processes. Small molecules may include any number of therapeutic agents presently known and used, or may be small molecules synthesized in a library of such molecules for the purpose of screening for function. Small molecules are distinguished from polymers and macromolecules by size and lack of polymerization. Small molecules may include peptides, peptoids and small organic molecules.

The candidate GSK3 kinase modulators and libraries of candidate GSK3 kinase modulators for screening by the methods of the invention may be derived from any of the various possible sources of candidate inhibitors, such as for example, libraries of peptides, peptoids, small molecules, and polynucleotides. The polynucleotide libraries may include antisense molecules or ribozymes. The GSK3 kinase modulators may be a polypeptide presented by phage display, provided mechanisms are designed to get the polypeptide modulator into the cell, or the polypeptide modulator was used to construct an intrabody or intracellular antibody. In general, a GSK3 kinase inhibitor may be any molecule that may be capable of inhibiting GSK3 activity and, in particular, that may be capable of enhancing an endothelial cell activity. In general, a GSK3 kinase activator may be any molecule that may be capable of activating GSK3 activity and, in particular, that may be capable of inhibiting an endothelial cell activity. Some libraries for screening may be subdivided into library pools for assaying modulation of GSK3 activity by the methods of the invention. Some of each pool is assayed and some is saved for reassay, or is further subdivided into subpools, should a positive be identified.

Further alternative agents include peptide analogs and derivatives that may act as stimulators or inhibitors of gene expression, or as ligands or antagonists. General means contemplated for the production of peptides, analogs or derivatives are known in the art (See, e.g., CHEMISTRY AND BIOCHEMISTRY OF AMINO ACIDS, PEPTIDES, AND PROTEINS—A SURVEY OF RECENT DEVELOPMENTS, Weinstein, B. ed., Marcell Dekker, Inc., publ. New York (1983). Moreover, substitution of D-amino acids for the normal L-stereoisomers may be carried out to increase the half-lives of the molecules.

Peptoids, polymers comprised of monomer units of at least some N-substituted moieties, may act as small molecule stimulators or inhibitors herein and may be synthesized as described in PCT 91/19735. Presently preferred amino acid substitutes are N-alkylated derivatives of glycine, which are easily synthesized and incorporated into polypeptide chains. However, any monomer unit that allows for the sequence specific synthesis of pools of diverse molecules is appropriate for use in producing peptoid molecules. The benefits of these molecules for the purpose of the invention is that they occupy different conformational space than a peptide and are more resistant to the action of proteases because their amide linkages are N-substituted.

Peptoids are easily synthesized by standard chemical methods. One method of synthesis is the “submonomer” technique described by R. Zuckermann et al., J. Am. Chem. Soc. 114:10646-7 (1992). Synthesis by solid phase techniques of heterocyclic organic compounds in which N-substituted glycine monomer units forms a backbone is described in copending application U.S. Pat. No. 5,977,301 or 5,877,278 entitled “Synthesis of N-Substituted Oligomers” filed on Jun. 7, 1995 and is herein incorporated by reference in full. Combinatorial libraries of mixtures of such heterocyclic organic compounds may then be assayed for the ability to alter gene expression.

Synthesis by solid phase of other heterocyclic organic compounds in combinatorial libraries is also described in U.S. Pat. No. 5,958,792, herein incorporated by reference in full. Highly substituted cyclic structures may be synthesized on a solid support by combining the submonomer method with powerful solution phase chemistry. Cyclic compounds containing one, two, three or more fused rings are formed by the submonomer method by first synthesizing a linear backbone followed by subsequent intramolecular or intermolecular cyclization as described in the same application.

Where the selected inhibitor of GSK3 kinase activity is a ribozyme, for example, a ribozyme targeting a GSK3 gene, the ribozyme may be chemically synthesized or prepared in a vector for a gene therapy protocol including preparation of DNA encoding the ribozyme sequence. The synthetic ribozymes or a vector for gene therapy delivery may be encased in liposomes for delivery, or the synthetic ribozyme may be administered with a pharmaceutically acceptable carrier. A ribozyme is a polynucleotide that has the ability to catalyze the cleavage of a polynucleotide substrate. Ribozymes for inactivating a gene may be prepared and used as described in Long et al., FASEB J. 7:25 (1993), and Symons, Ann. Rev. Biochem. 61:641 (1992), Perrotta et al., Biochem. 31:16, 17 (1992); and U.S. Pat. No. 5,225,337, U.S. Pat. No. 5,168,053, U.S. Pat. No. 5,168,053 and U.S. Pat. No. 5,116,742, Ojwang et al., Proc. Natl. Acad. Sci. USA 89:10802-10806 (1992), U.S. Pat. No. 5,254,678 and in U.S. Pat. No. 5,144,019, U.S. Pat. No. 5,225,337, U.S. Pat. No. 5,116,742, U.S. Pat. No. 5,168,053. Preparation and use of such ribozyme fragments in a hammerhead structure are described by Koizumi et al., Nucleic Acids Res. 17:7059-7071 (1989). Preparation and use of ribozyme fragments in a hairpin structure are described by Chowrira and Burke, Nucleic Acids Research 20:2835 (1992).

The hybridizing region of the ribozyme or of an antisense polynucleotide may be modified by linking the displacement arm in a linear arrangement, or alternatively, may be prepared as a branched structure as described in Horn and Urdea, Nucleic Acids Res. 17:6959-67 (1989). The basic structure of the ribozymes or antisense polynucleotides may also be chemically altered in ways quite familiar to those skilled in the art. Chemically synthesized ribozymes and antisense molecules may be administered as synthetic oligonucleotide derivatives modified by monomeric units. Ribozymes and antisense molecules may also be placed in a vector and expressed intracellularly in a gene therapy protocol.

The invention includes generating mRNA and CDNA libraries for screening for modulation of GSK3 kinase activity, may require overexpression of recombinant GSK3, and may also involve transforming a cell with the gene for GSK3 for expression in the assay. Exemplary systems for generating polypeptides or libraries useful for the method of the invention would include, for example, any standard or useful mammalian, bacterial, yeast or insect expression system, many of which are described in WO 96/35787. Thus, any polypeptide or peptide useful in the invention may be made by these or other standard methods.

Other items not specifically exemplified, such as plasmids, may be constructed and purified using standard recombinant DNA techniques described in, for example, Sambrook et al. (1989), MOLECULAR CLONING, A LABORATORY MANUAL, 2d edition (Cold Spring Harbor Press, Cold Spring Harbor, N.Y.), and Ausubel et al., CURRENT PROTOCOLS IN MOLECULAR BIOLOGY (1994), (Greene Publishing Associates and John Wiley & Sons, New York, N.Y.) under the current regulations described in United States Dept. of HHS, NATIONAL INSTITUTE OF HEALTH (NIH) GUIDELINES FOR RECOMBINANT DNA RESEARCH. These references include procedures for the following standard methods: cloning procedures with plasmids, transformation of host cells, cell culture, plasmid DNA purification, phenol extraction of DNA, ethanol precipitation of DNA, agarose gel electrophoresis, purification of DNA fragments from agarose gels, and restriction endonuclease and other DNA-modifying enzyme reactions.

In one embodiment, the screening assay involves determining whether a putative angiogenesis modulator modulates apoptotic cell death of endothelial cells. The method involves contacting a putative angiogenesis modulator with the cells of the test sample under conditions to permit entry of the agent into the cell, determining a test sample index cell number, inducing apoptotic cell death in a test sample containing an endothelial cell in the presence of the putative angiogenesis modulator and comparing the test sample index cell number with a control index cell number of a control sample. The control sample contains cells that have been contacted with an angiogenesis promoter of the invention under conditions to permit entry of the angiogenesis promoter into the cells, and their index cell number is used as a reference number. The index cell number of the test sample as compared with the corresponding index cell number of the control sample is indicative of the inhibitory activity of the putative angiogenesis promoter in inhibiting death of the endothelial cells.

In one embodiment, the foregoing screening assay occurs in vitro. In some preferred embodiments, the cells are vascular endothelial cells.

In another embodiment, the foregoing screening assay occurs in vivo. In some preferred embodiments, the cells are cells of a subject from a tissue selected from the group consisting of myocardium, skeletal musculature and vascular endothelium.

Cell death may be induced in a variety of ways well known in the art, including administration of glucocorticoids, reduction of hormone and/or growth factor levels, chemotherapy (toxic agents), mechanical injury and DNA damage.

The index cell number of the test sample as compared with the equivalent index cell number of the control sample serves as an indicator of the properties of the test agent in inhibiting death of the cells. An “index cell number” refers to a number of viable cells, to a number of dead cells, or to percentages of the foregoing numbers in relation to a total number of cells in a sample. Stains specific for either viable cells or dead cells may be used in order to facilitate the cell counting. Such stains are well known in the art, and exemplary ones are described below in the Examples. An index cell number, for example, of viable cells in the test sample, would be the “equivalent index cell number” of viable cells in the control sample.

The invention will be more fully understood by reference to the following examples. These examples, however, are merely intended to illustrate the embodiments of the invention and are not to be construed to limit the scope of the invention.

### EXAMPLES

### Example 1

### Glycogen Synthase Kinase-3β Functions in Endothelial Cells to Negatively Regulate Angiogenesis

Introduction to Example 1. Glycogen synthase kinase-3β (GSK3β) plays important roles in metabolism, embryonic development and tumorigenesis. Here we investigated the role of GSK3β signaling in vascular biology by examining its function in endothelial cells (EC). In EC, the regulatory phosphorylation of GSK3β was found to be under the control of PI 3-kinase-, MAPK- and protein kinase A-dependent signaling pathways. Transduction of a non-phosphorylatable, constitutively-active mutant of GSKβ promoted apoptosis under conditions of prolonged serum deprivation or the disruption of cell-matrix attachments. Conversely, transduction of catalytically-inactive GSK3β promoted EC viability under conditions of cellular stress. Under normal cell culture conditions, activation of GSK3β signaling inhibited migration of EC to VEGF or bFGF. Angiogenesis was inhibited by GSK3β activation in an in vivo matrigel plug assay, whereas inhibition of GSK3β signaling enhanced capillary formation. These data suggest that GSK3β functions at the nodal point of converging signaling pathways to regulate angiogenesis through its control of vascular cell migration and viability.

In this study, we initially examined the upstream signaling pathways that control GSK3β phosphorylation and inactivation in EC. To determine the functional significance of this signaling pathway, GSK3 activity was modulated using adenoviral vectors expressing mutant GSK3 proteins and effects on viability, migration and angiogenesis were assessed. The results of these experiments are described below.

Materials. LY294002 was purchased from Cell Signaling Technology (St. Louis, Mo.). PD98059, SB203580, bisindolylmaleimide I, H-89, 8-bromo 3,5-cyclic AMP, forskolin, and 3-isobutyl-1-methyxanthine which were purchased from Calbiochem (San Diego, Calif.). Recombinant human VEGF165, basic FGF157 and PDGF-BB were purchased from R & D systems (Minneapolis, Minn.).

Cell culture and adenoviral vectors. Human umbilical vein ECs (HUVECs) were isolated as previously described (29) and cultured in EGM media (Clonetics, Walkersville, Md.). Four to six passage cells were used in this study. To examine the regulation of GSK3β phosphorylation, HUVECs were serum-starved for 15 hours, treated with the indicated agents for 1 hour, and stimulated with 10% fetal bovine serum. To examine serum deprivation-induced apoptosis, HUVECs were transduced with the indicated adenoviral construct and cultured in serum-free media for 1 to 4 days. Some assays employed a replication-defective adenoviral vector expressing catalytically-inactive GSK313 (GSK3β-KM) where lysine residues at positions 85 and 86 were mutated to methionine and alanine, respectively (30, 31). Another vector expressed the nonphosphorylatable, constitutively-active mutant of GSK3β (GSK3β-S9A) where the serine residue at position 9 was mutated to alanine (30, 31). As a control, an adenovirus vector expressing β-galactosidase gene was used (32). To examine the effect of GSK-3β signaling on anoikis, HUVECs were trypsinized and seeded on the poly 2-hydroxy ethyl methacrylate (HEMA)-coated dishes in the EGM media. Poly-HEMA solution (10 mg/ml) (Sigma, St. Louis, Mo.), attachment inhibitor, was dispensed to cover the entire surface of 10 cm dish or each well of 6-well plate, and completely dried in a culture hood as described previously (33). This process was repeated twice and then the treated surface was extensively washed by phosphate buffered saline (PBS) before use for suspension cultures. At a serial time points after suspension, HUVECs were harvested for immunoblot or viability assays, or were transferred to adhesive plate and cultured to evaluate the reattachment and growth of the anchorage-deprived cells.

Immunoblot analysis. HUVECs were washed in phosphate-buffered saline and harvested by scraping in 50 mM Tris-HCl (pH 7.2), 250 mM NaCl, 1% NP40, 0.05% SDS, 2 mM EDTA, 0.5% deoxycholic acid, 10 mM 13-glycerophosphate, 1 mM vanadate, 1 mM phenyl methyl sulfonyl fluoride (PMSF). One mini tablet of protease inhibitor cocktail (Roche, Summerville, N.J.) per 10 ml of lysis buffer, vanadate, and PMSF were added just prior to use. Protein concentration was determined with protein assay kit (Pierce, Rockford, Ill.). Protein (20 μg) was separated on SDS-polyacrylamide electrophoresis gel and transferred to a polyvinylidene difluoride membrane (Millipore, Bedford, Mass.). The membrane was blocked with T-PBS (1×PBS, 0.3% Tween-20) containing 3% dry milk and incubated with primary antibody overnight at 4° C. After three washes with T-PBS, the membrane was reblocked and incubated with secondary antibody for 1 hour at room temperature. ECL or ECL-PLUS (Amersham, Piscataway, N.J.) was used for detection. To reprobe the membrane, it was treated with Restore Western Blot stripping buffer (Pierce, Rockford, Ill.). The primary antibodies used were anti-phospho GSK-3β (Ser9) antibody (1:750 dilution, Cell Signaling Technology, Beverly, Mass.), anti-phospho Akt (Ser473) antibody (1:250 dilution, Cell Signaling Technology, Beverly, Mass.), anti-total GSK-3β antibody (1:1000 dilution, Santa Cruz, Santa Cruz, Calif.), and anti-a tubulin antibody (1:4000 dilution, Oncogene, Boston, Mass.). The secondary antibodies were anti-rabbit IgG/HRP conjugate or anti-mouse IgG/HRP conjugate (1:2500 dilution, Promega, San Luis Obispo, Calif.).

Migration assays. Migration assays were performed as described previously using a modified Boyden chamber (Neuroprobe, Gaithersburg, Md.) (34). HUVECs were infected with adenoviruses overnight in EGM media, then serum-starved for 5 hours in EBM media (Clonetics, Walkersville, Md.), and trypsinized. Cells were resuspended in EBM media as 100,000 cells/300 □l, and were added on the upper chamber. VEGF (50 ng/ml) or basic FGF (25 ng/ml) in EBM were added into the lower chamber. For migration assays of human aortic smooth muscle cells (HAoSMC), PDGF (50 ng/ml) or basic FGF (50 ng/ml) was used. Polycarbonate filters (8 μm pores; Neuroprobe, Gaithersburg, Md.) precoated with gelatin by overnight incubation in 0.5% gelatin solution, was set between lower and upper chambers. The chamber was incubated for 5 hours at 37° C. Then filter was carefully removed and cells attached on the upper side were wiped off. HUVECs migrating through the filter and appearing on the lower side were fixed by careful immersion of the filter into 70% ethanol for 15 minutes, stained with Giemsa solution and counted in three random fields per well. Each experiment was performed in duplicate and four separate experiments were performed.

Cell viability assays. HUVECs in 96-well plates were infected with adenovirus and analyzed using tetrazolium salt WST-1 as instructed by manufacturer (Roche, Summerville, N.J.). DNA fragmentation was assessed by flow cytometry. For these assays, HUVECs were infected with adenoviruses and serum-starved for 2-4 days. At several time points after serum-starvation, the attached and floating HUVECs were harvested and fixed in cold 90% ethanol for 20 minutes and then resuspended in staining buffer consisting of 1 mg/ml RNaseA, 20 μg/ml propidium iodide and 0.01% NP40. DNA content was analyzed by flow cytometry on FL-2 channel and gating was set to exclude debris and cellular aggregates. For each analysis, 10,000 events were counted. Alternatively, pyknotic nuclei were assessed by Hoechst staining. For these assays, HUVECs were fixed with 4% paraformaldehyde for 30 minutes in room temperature, carefully washed with PBS twice, and stained with 10 mg/ml solution of Hoechst 33342 (Sigma, St. Louis, Mo.) at room temperature with light shielded. Cell viability was also assessed by annexin V/propidium iodide double staining. For these experiments serum-deprived HUVECs were cultured in 4-well slide chambers (Nunc). Media was removed and cells were covered with staining solution that contains annexin-V-fluorescein, propidium iodide, and binding buffer (Roche, Summerville, N.J.) for 15 minutes at room temperature. To examine the anchorage deprivation-induced apoptosis, HUVECs in suspension culture were centrifuged at 200g for 5 minutes. The cell pellet was resuspended in staining solution, incubated for 15 minutes at room temperature, and spread on slide glass with cover.

In vivo angiogenesis assay. The formation of new vessels in vivo was evaluated by matrigel plug assay as described previously (35-37). For these experiments, equal amounts of heparin (10 units/ml) and basic FGF (1 μg/ml) (R & D) were mixed, and 5 μl this solution was mixed on ice with 10 ml of matrigel (Becton Dickinson, San Jose, Calif.) for a final concentration of basic FGF to be 250 ng/ml. Solutions of adenoviral vectors encoding β-gal, GSK3β-KM, or GSK3β-S9A mixed in with matrigel solution on ice (2×10⁸ plaque forming unit of virus (10 μl) should be contained in 500 μl). Five hundred μl of matrigel containing growth factor and adenovirus was injected subcutaneously near the right mid abdomen of C57BL mice (Jackson Laboratories, Bar Harbor, Me.). Mice were sacrificed 10 days after the injection. The matrigel plugs with the adjacent subcutaneous tissues were carefully recovered by en bloc resection, embedded in OCT compound, and quick-frozen in liquid nitrogen. Immunohistochemistry for hemagglutinin (HA) or CD31 (PECAM-1), and histochemistry for alkaline phosphatase were performed on adjacent frozen sections. The primary antibodies were anti-HA rabbit polyclonal antibody (1:20, Santa Cruz, Santa Cruz, Calif.) and anti-PECAM-1 goat polyclonal antibody (1:20 dilution, Santa Cruz, Santa Cruz, Calif.). The secondary antibodies were biotinylated horse anti-goat IgG antibody (1:100 dilution, Vector) and biotinylated goat anti-rabbit IgG antibody (1:100 dilution, Vector). Other components for immunohistochemistry were from LSAB-2 kit (Dako, Carpinteria, Calif.).

Statistical analysis. Data are shown as mean±S.E.M. All data were evaluated with a two tailed, paired T test or compared by one-way analysis of variance.

Results:

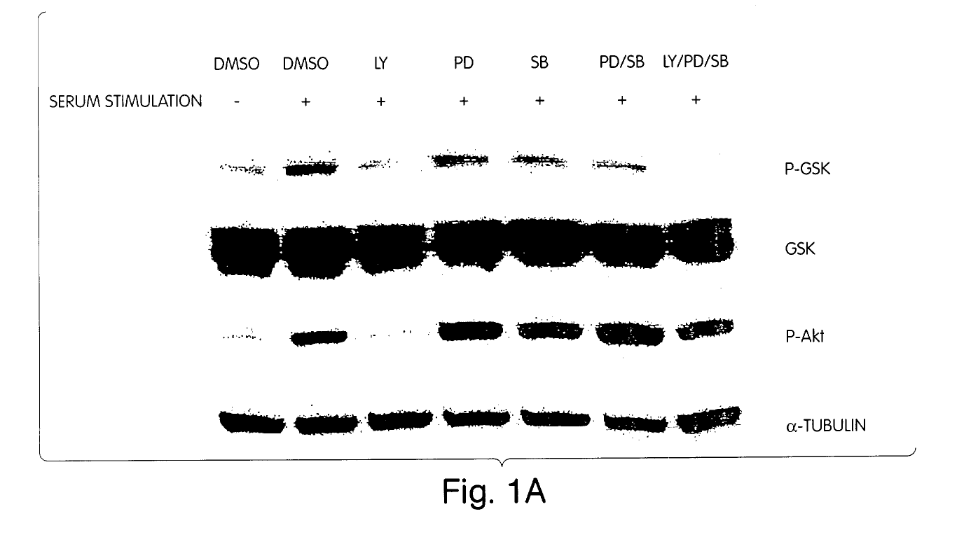

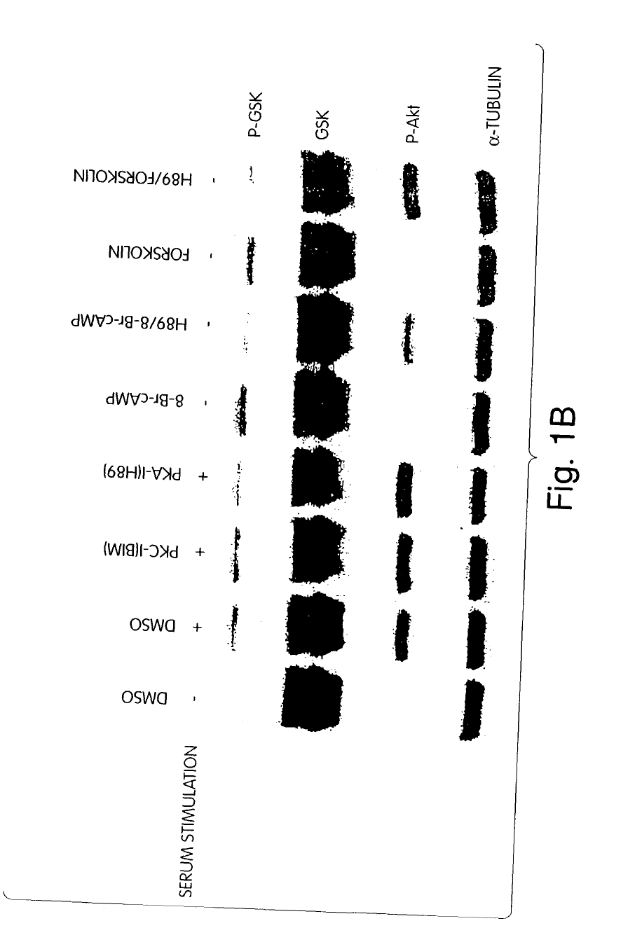

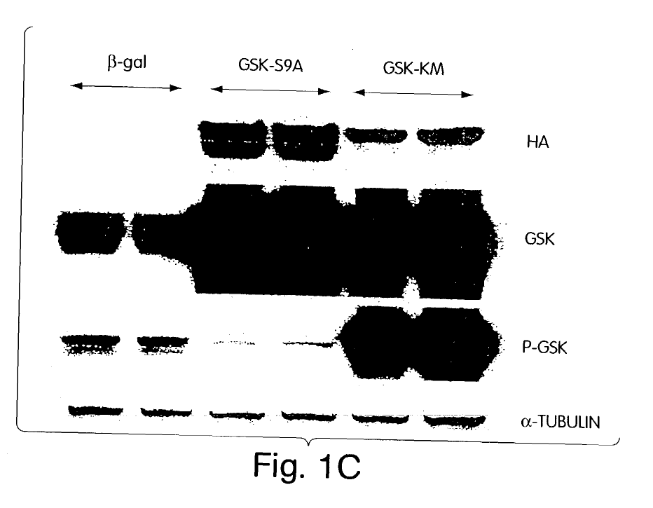

Multiple signal transduction pathways regulate GSK-3β in EC. We first assayed for the presence of GSK3β in EC and examined the signal transduction pathways that are involved in its regulatory phosphorylation at serine 9. Stimulation of serum-deprived HUVECs with 10% FBS for 1 hour led to a marked increase in GSK3β phosphorylation (FIG. 1A). The phosphorylation of GSK3β was paralleled by an increase in the phosphorylation of Akt, a candidate upstream kinase that is regulated by PI3K (5, 23). The serum-induced phosphorylation of GSK3β was blocked by pretreatment with the PI3K inhibitor LY294002 and partially inhibited by pretreatment with the MAPK inhibitors PD98059 and/or SB203480. Akt phosphorylation was blocked by LY294002, but not by PD98059 or SB203480. Furthermore, the combined administration of PD98059 and SB203480 detectably elevated the level of Akt phosphorylation in the presence or absence of PI3K inhibition. In contrast, MAPK and PI3K inhibition had additive effects in reducing GSK3β phosphorylation.

PKA is reported to regulate GSK3β in fibroblast, epithelial, or neuronal cells (6, 24). In HUVECs, the PKA inhibitor H89 inhibited the serum-induced phosphorylation of GSK 3β (FIG. 1B). Stimulation of serum-deprived HUVECs with the PKA activators 8-bromo cAMP or forskolin induced phosphorylation of GSK3β and this induction was blocked by pretreatment with H89 (FIG. 1B). Conversely, activation of PKA diminished Akt phosphorylation, whereas treatment with H89 promoted Akt phosphorylation. Collectively, these data show that although PI3K signaling participates in GSK3β regulation, Akt and GSK3β phosphorylation are differentially regulated by MAPK and PKA signaling pathways.

To modulate the intracellular GSK3β activity, replication-defective adenoviral vectors that express either a non-phosphorylatable, constitutively-active mutant (GSK3β-S9A) or a catalytically-inactive mutant (GSK3β-KM) that functions as a dominant-negative (30, 31) were employed. As shown in FIG. 1C, HUVECs transduced with GSK3β-S9A showed a decline in phosphorylated GSK3β, indicative of an activation of GSK 3β signaling. In contrast, transduction with GSK3β-KM led to an increase in GSK3β protein that was phosphorylated at serine 9.

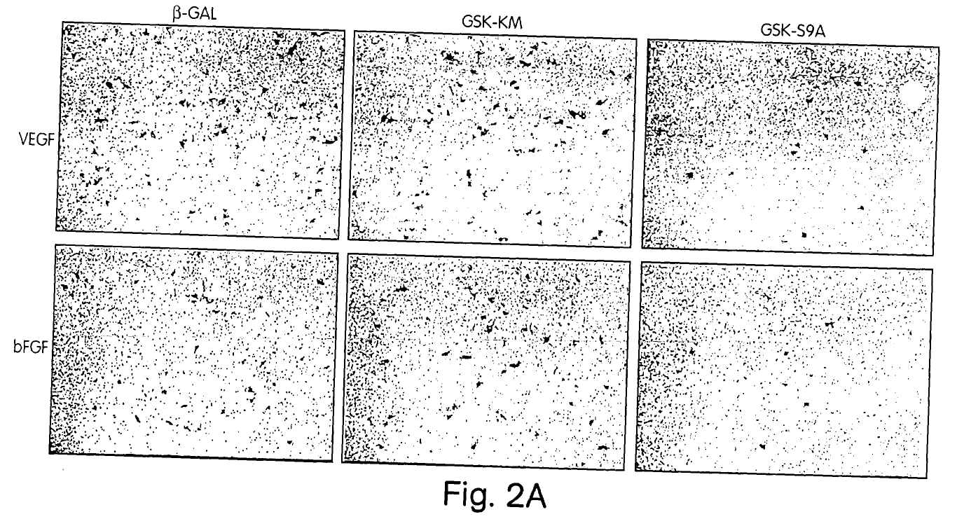

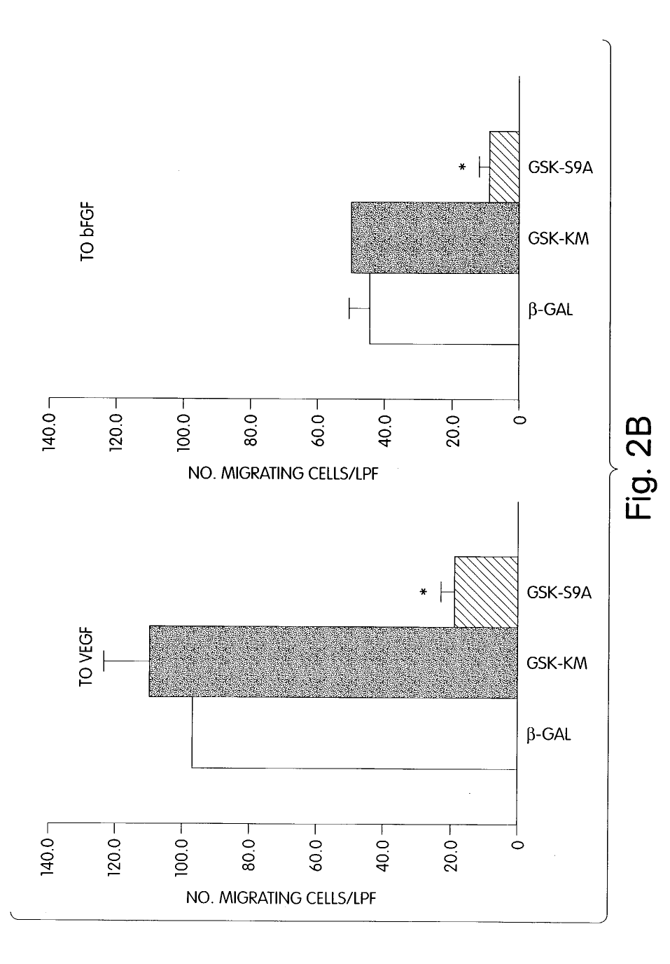

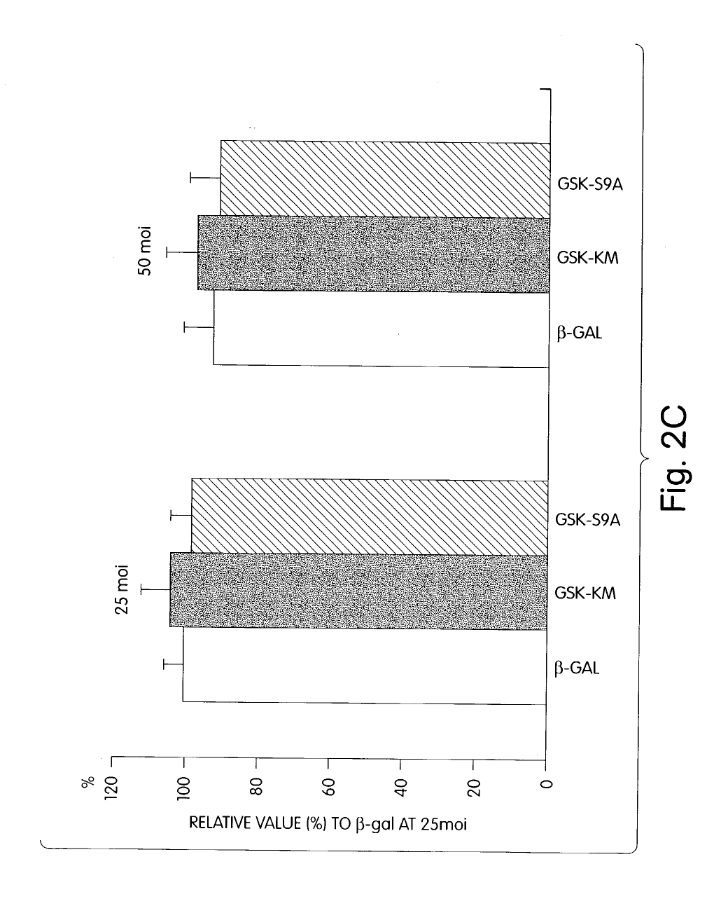

Role of GSK3β in vascular cell migration. When HUVECs were transduced with GSK3β-S9A (50 multiplicity of infection), their chemotactic activities toward VEGF or bFGF were significantly decreased (FIG. 2A, B). Under the conditions of these assays, VEGF was a more potent chemoattractant than bFGF. In contrast, transduction of GSK3β-KM slightly enhanced the directional migration of HUVECs toward VEGF or bFGF, but this was not statistically significant. The antimigratory effect of GSK3β-S9A was not due to a cytotoxic effect of this vector because Adeno-GSK3β-S9A did not decrease viability as assessed by a WST-1 assay under these cell culture conditions (FIG. 2C). At late time points of serum deprivation (>2 days) modulation of GSK3β signaling influenced cellular viability (see below).

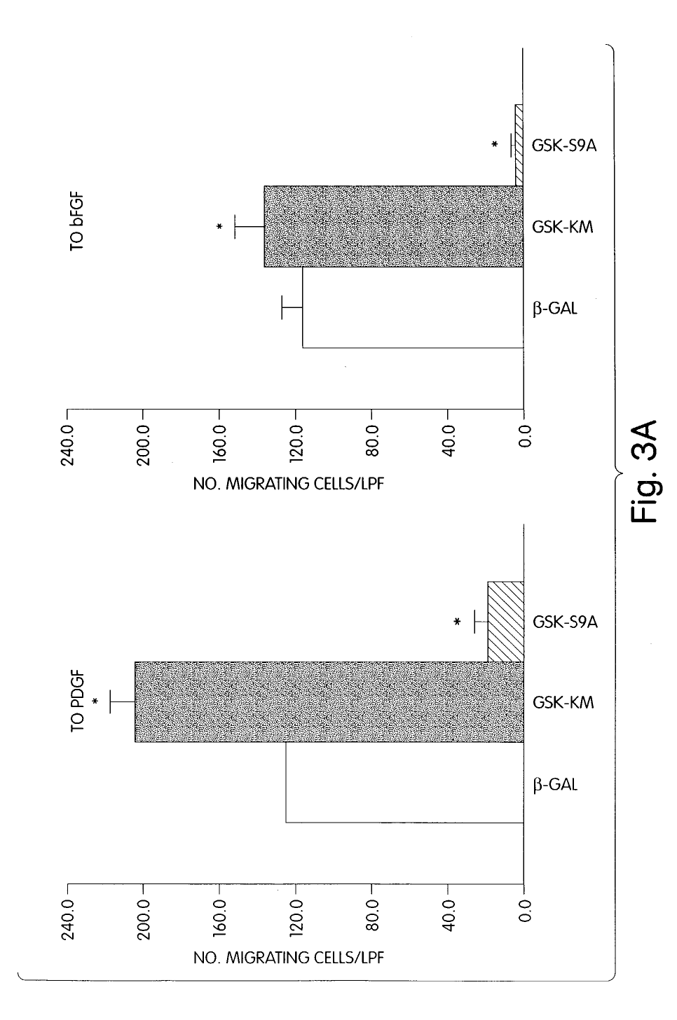

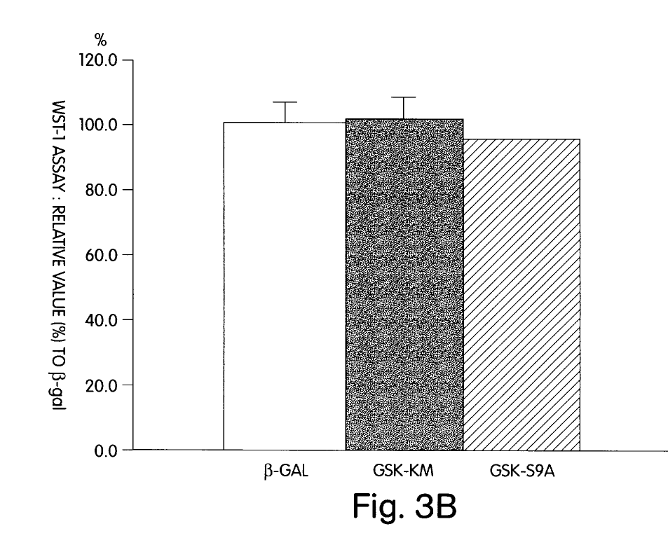

Because the role of GSK3β on the migration of vascular cells had not been described previously, we assayed the effects of GSK3β-S9A and GSKβ-KM on the migration of human aortic vascular smooth muscle cells (HAOSMC) toward bFGF or PDGF. Transduction of GSK3β-S9A significantly inhibited HAoSMC's chemotactic activities toward PDGF or bFGF in the Boyden chamber analysis (FIG. 3A). Conversely, transduction of GSK3β-KM significantly enhanced the chemotaxis of HAoSMC toward either growth factor. Under these experimental conditions, GSK3β-S9A or GSK3β-KM did not show a significant cytotoxic or cytoprotective effect, respectively, as assessed by WST-1 assay (FIG. 3B).

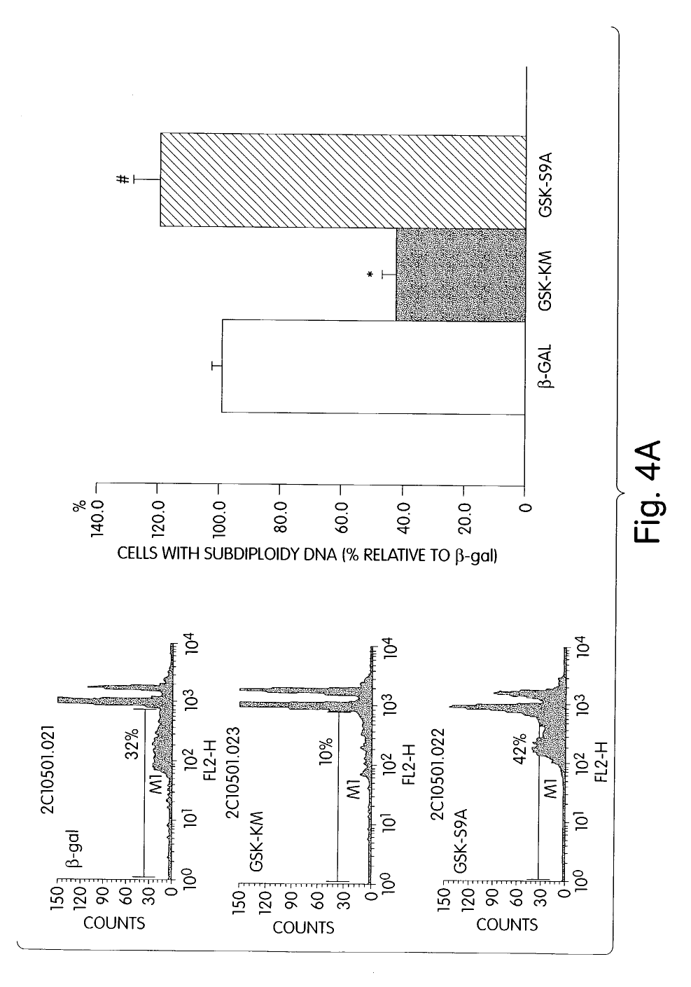

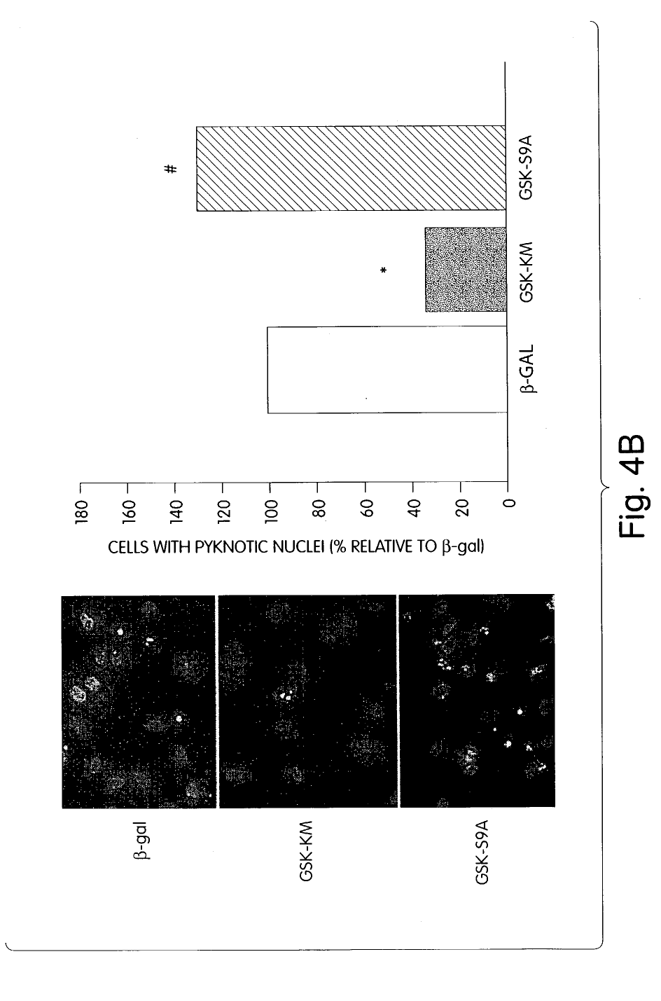

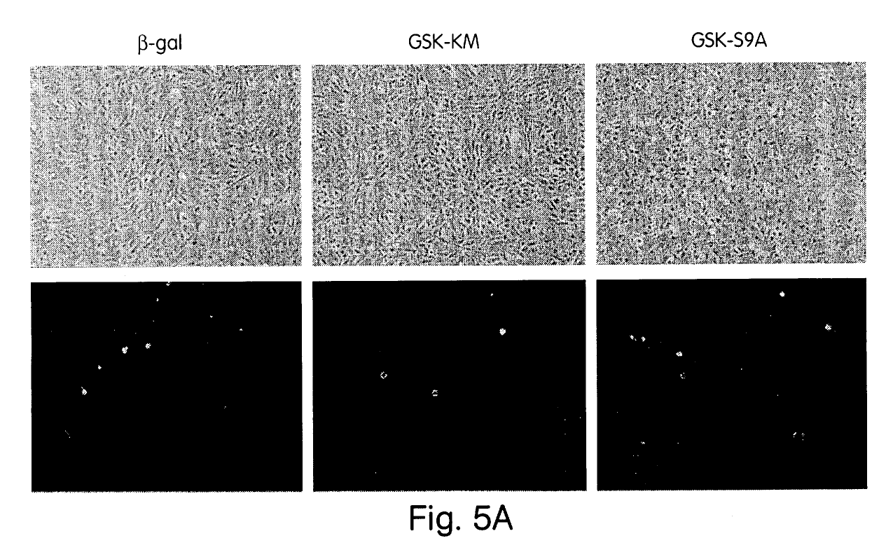

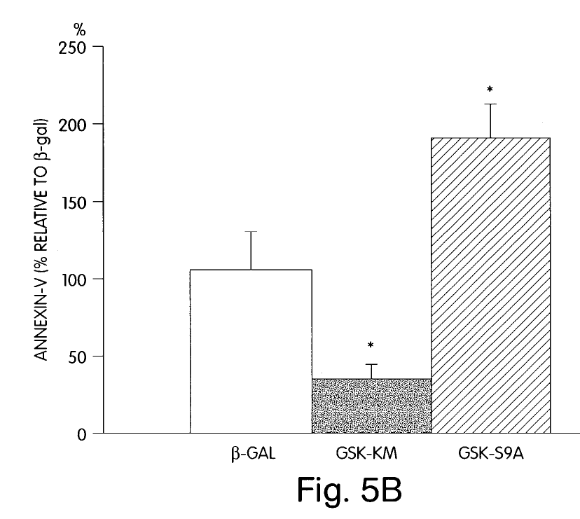

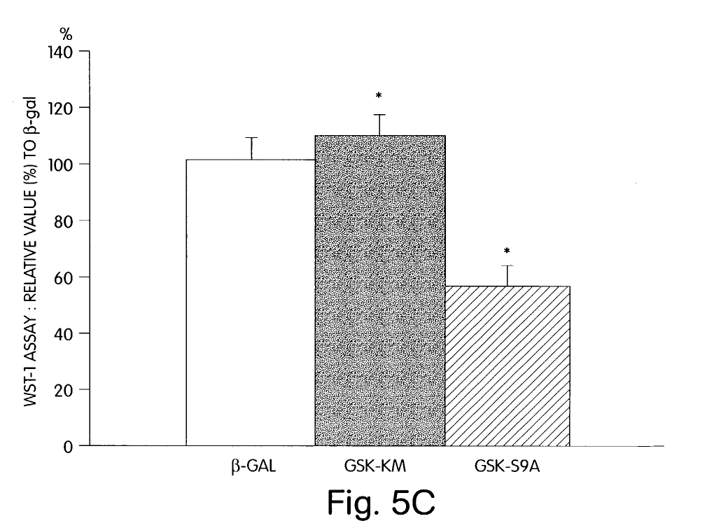

GSK 3β signaling controls serum-deprivation-induced apoptosis of EC. As shown above, transduction of GSK3β-S9A or GSK3β-KM had no effect on cell viability under normal cell culture conditions with mitogens present in the media or after short periods of serum deprivation. Therefore, to examine the role of GSK3β signaling in EC viability, HUVECs were infected with the adenoviral vectors expressing GSK3β mutants and incubated in serum-free media for 4 days to promote apoptosis (38). Under these culture conditions, transduction of GSK3β-KM significantly reduced the subdiploid fraction of DNA detected by FACS analysis, whereas the constitutively-active GSK3β-S9A increased DNA degradation (FIG. 4A). These data were corroborated by assessing the impact of GSK3β signaling modulation on the frequency of pyknotic nuclei in Hoechst 33342-stained HUVEC cultures (FIG. 4B). Plasma membrane phospholipid asymmetry, another marker of apoptosis, was also assessed by analyzing the frequency of annexin V-positive cells (FIG. 5A, B). Transduction of serum-deprived HUVEC with GSK3β-KM reduced the frequency of annexin V-positive cells. GSK3β-KM also reduced the frequency of annexin V-positive cells that stained positive with propidium iodide, which marks the later phases of cell death. Conversely, transduction of GSK3-S9A increased the frequencies of cells that stained positive for annexin V or propidium iodide. Consistent with these data, transduction of GSK3β-KM significantly promoted viability as assessed by the WST-1 assay of mitochondrial function, whereas GSK3β-S9A reduced mitochondrial function (FIG. 5C).

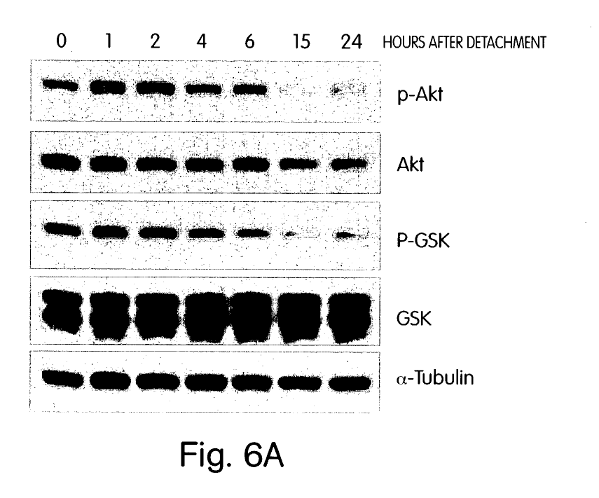

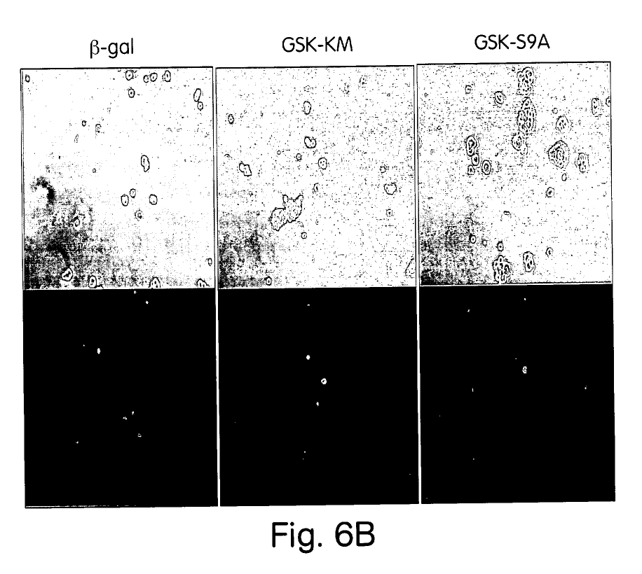

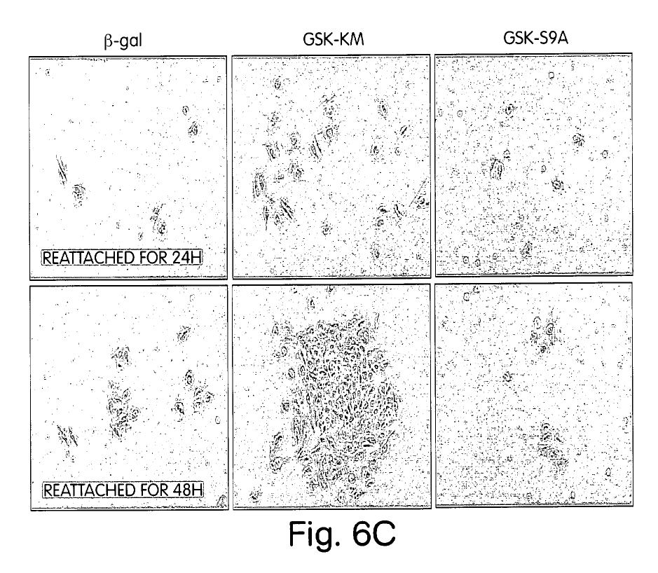

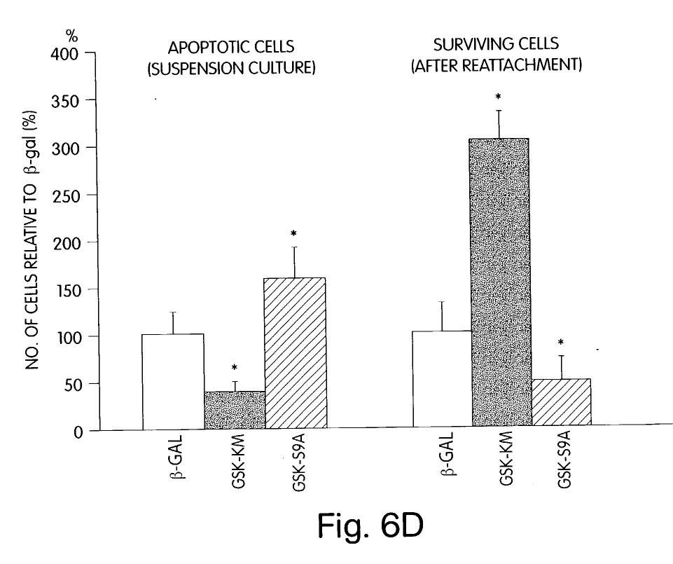

GSK3β regulates EC anoikis. Adhesion to extracellular matrix is an important determinant of EC survival under conditions of neovascularization (39). Thus, the role of GSK3β signaling in HUVEC anoikis was assessed. As shown in FIG. 6A, there was a time-dependent decrease in phosphorylated GSK3β following the placement of HUVEC in suspension culture. The decrease in GSK3β phosphorylation was paralleled by a decrease in the phosphorylation of Akt, a regulator of EC anoikis (38). To assess the role of GSK3β signaling in EC anoikis, HUVECs were transduced with GSK3β-KM or GSK3β-S9A prior to placement in suspension cultures. Annexin V staining revealed that expression of GSK3β-KM protected HUVECs from anoikis compared to control HUVECs that were transduced with β-galactosidase (FIG. 6B, D). Conversely, GSK-S9A promoted cell death under these conditions. These findings were confirmed by a reattachment assay where HUVECs were reseeded on the adhesive plate after 1 day of the suspension culture (FIG. 6C, D). Cells expressing GSK-KM displayed a higher frequency of successful reattachment than control cells, whereas HUVEC transduced with GSK-S9A displayed a lower frequency of reattachment following incubation in suspension culture.

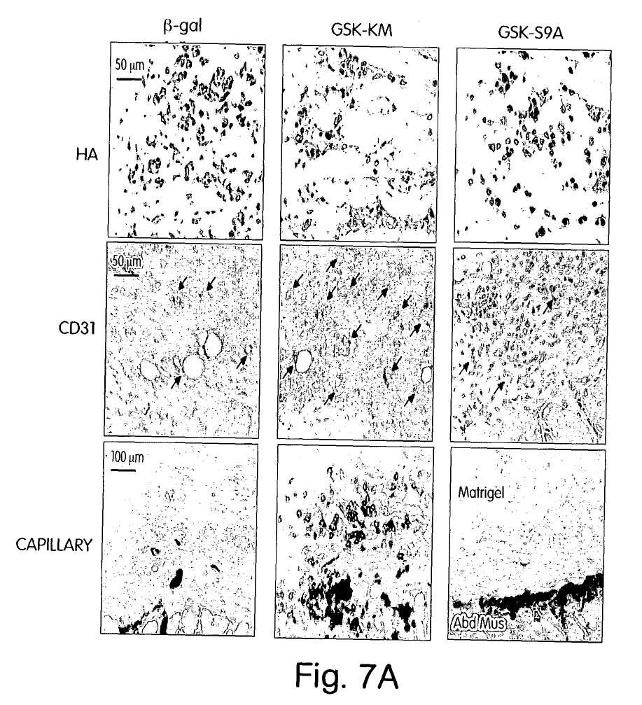

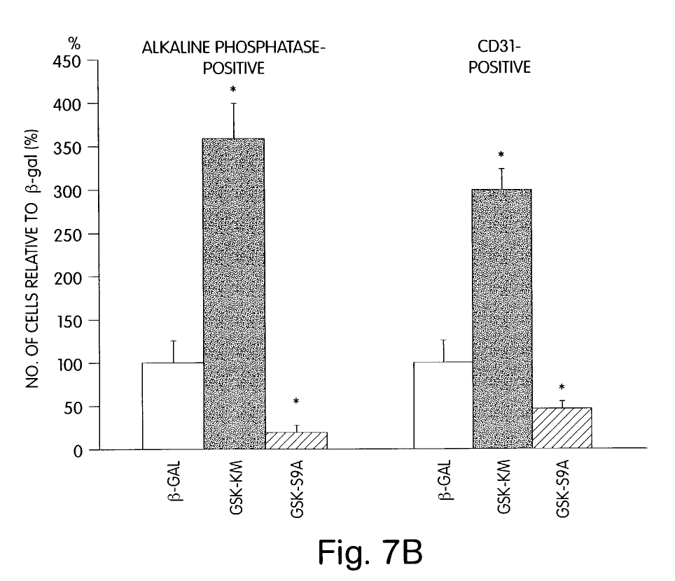

GSK3β regulates angiogenesis. A matrigel plug assay in mice was employed to test the role of GSK3β signaling in angiogenesis in vivo. Adenoviral vectors (2×10⁸ PFU) were incorporated in the matrigel plugs along with bFGF (250 ng/ml) prior to subcutaneous implantation in the abdomen of C57BL6 mice for 10 days prior to recovery. Expression of the HA-tagged GSK3β transgene products was confirmed by immunohistochemistry (FIG. 7A). HA-positive immunostaining was detectable in plugs formulated with adenoviral vectors encoding GSK-KM and GSK-S9A, but little or no signal was detected in plugs formulated with the β-galactosidase-expressing adenovirus. EC infiltration of these plugs was assessed by immunohistochemical analysis of CD31-positive cells. Plugs formulated with Adeno-GSK3β-KM exhibited significantly higher densities of EC than control plugs (FIG. 7A, B). Conversely, plugs formulated with GSK3β-S9A displayed a lower density of CD31-positive cells than control. These data were corroborated by analyzing the densities of alkaline phosphatase-positive capillaries within these plugs (FIG. 7A, B).

Discussion:

This study examined the regulation and function of GSK3β in EC biology and blood vessel growth. Specific kinase inhibitors were used to assess the signaling pathways that regulate GSK3β in EC. The strong inhibitory effect of LY294002 on GSK3β phosphorylation suggests that PI3K-dependent pathways are a major regulator of its activity in response to mitogen stimulation, and these data are consistent with the finding that GSK3β is directly phosphorylated by the PI3K-regulated protein kinase Akt (5, 22, 23). However, GSK3β does not function solely as an obligate downstream intermediate of PI3K/Akt signaling. It is shown that MAPK inhibition downregulates GSK3β phosphorylation, while it promotes Akt phosphorylation. Furthermore, PKA agonists were found to increase GSK3β phosphorylation, but have the opposite effect on Akt. Collectively, these data suggest that GSK3β signaling functions at a step that is central to many angiogenesis-regulatory pathways in EC, and thus its activity may be controlled by a variety of angiogenic growth factors and hormones.

EC survival is an important factor influencing angiogenesis and vessel integrity, and angiogenic growth factor withdrawal will lead to vessel regression in retina and tumors (40-42). Previous studies have found PI3K/Akt signaling mediates EC survival in response to angiogenic growth factors, including VEGF and angiopoeitin-1 (38, 43-45). This study extends these prior observations by documenting that GSK3β plays a key role in controling EC survival in response to growth factor limitation. Activation of GSK3β signaling increased EC apoptosis in response to growth factor deprivation as shown by increased DNA fragmentation, decreased mitochondrial function and a loss of phospholipid asymmetry. Conversely, ablation of GSK3β signaling protected cells from apoptosis under conditions of mitogen-deprivation. Although growth factors initiate the angiogenic process, proper associations between cells and matrix are essential for neovascularization because they promote EC survival as they migrate toward the angiogenic source (39). Previous studies have shown that the pro-survival signals from cell surface-extracellular matrix interactions can be mediated by Akt (38) and by ILK (46), which phosphorylates both Akt and GSK3β (47). When EC were deprived of anchorage attachments, there were time-dependent decreases in the levels of phosphorylated GSK3β as well as Akt. Apoptosis under these conditions was markedly reduced by the expression of catalytically-inactive GSK3β, providing direct evidence that GSK3β is an important component of this survival pathway. Collectively, these data reinforce the notion that GSK3β operates at the convergence of multiple signaling pathways to regulate EC survival in response to diverse external stresses.

This study also shows that GSK313 signaling controls EC migration toward VEGF or bFGF and smooth muscle cell migration toward PDGF or bFGF. These effects on migration were not due to cytotoxic or cytoprotective actions of the different GSK3β vectors because these assays were performed during short periods of serum deprivation where apoptosis is minimal. Because the migration and survival of vascular cells are essential components of the angiogenic response, a mouse angiogenesis assay was employed to assess the consequences of adenovirus-mediated GSK3β gene transfer on capillary infiltration of matrigel plugs containing bFGF. In these experiments, the kinase mutant GSK3β increased capillary density in the plug, whereas constitutively-active GSK3β markedly reduced capillary formation. From these experiments, we conclude that GSK3β signaling functions in EC to negatively regulate angiogenesis.

Previous studies have shown that Akt regulates angiogenic cellular responses in EC including survival (38, 43), migration (48) and NO production (49, 50). Moreover, adenovirus-mediated transfer of a constitutively-active Akt gene to the vascular endothelium of ischemic limbs will promote collateral vessel formation and improve perfusion (51). The results of this study increase our knowledge of this regulatory pathway by documenting that GSK3β, one of many substrates for the Akt protein kinase, may regulate EC survival and migration in vitro and angiogenesis in vivo. Furthermore, it is shown that MAPK- and PKA-dependent signaling pathways promote changes in GSK3β phosphorylation that favor angiogenesis. These observations are significant because some angiogenic factors, such as bFGF, are efficient activators of MAPK, but do not activate PI3K/Akt signaling (data not shown). Consistent with the notion that distinct angiogenic signals converge on GSK3β, its phosphorylation is also controlled by Wnt through a mechanism distinct from that employed by mitogenic factors (1, 20, 26), and recent studies have shown that Wnt can regulate EC growth (52). Thus, GSK3β may control blood vessel growth by functioning at a nodal point of multiple-signaling pathways where it coordinates EC responses to both pro- and anti-angiogenic inputs.

### BACKGROUND OF THE INVENTION AND EXAMPLES REFERENCE LIST

1. Kim, L., and A. R. Kimmel. 2000. GSK3, a master switch regulating cell-fate specification and tumorigenesis. Curr. Opin. Genet. Dev. 10:508-514.

2. Welsh, G. I., C. Wilson, and C. G. Proud. 1996. Gsk3: a SHAGGY frog story. Trends Cell. Biol. 6:274-279.

3. Woodgett, J. R. 1991. A common denominator linking glycogen metabolism, nuclear oncogenes and development. Trends Biochem. Sci. 16:177-181.

4. Dajani, R., E. F. Fraser, S. M. Roe, N. Young, V. Good, T. C. Dale, and L. H. Pearl. 2001. Crystal structure of glycogen synthase kinase 3b: Structural basis for phosphate-primed substrate specificity and autoinhibition. Cell. 105:721-732.

5. Cross, D. A. E., D. R. Alessi, P. Cohen, M. Andjelkovic, and B. A. Hemmings. 1995. Inhibition of glycogen-synthase kinase-3 by insulin mediated by protein-kinase-B. Nature. 378:785-789.

6. Fang, X., S. X. Yu, Y. Lu, R. C. Bast, J. R. Woodgett, and G. B. Mills. 2000. Phosphorylation and inactivation of glycogen synthase kinase 3 by protein kinase A. Proc. Natl. Acad. Sci. USA. 97:11960-11965.

7. Hoeflich, K. P., J. Luo, E. A. Rubie, M. S. Tsao, O. Jin, and J. R. Woodgett. 2000. Requirement for glycogen synthase kinase-3beta in cell survival and NF-kappaB activation. Nature. 406:86-90.

8. Skurat, A. V., and P. J. Roach. 1995. Phosphorylation of sites 3a and 3b (Ser640 and Ser644) in the control of rabbit muscle glycogen synthase. J. Biol. Chem. 270:12491-12497.

9. Yost, C., M. Torres, J. R. Miller, E. Huang, D. Kimelman, and R. T. Moon. 1996. The axis-inducing activity, stability, and subcellular distribution of b-catenin is regulated in Xenopus embryos by glycogen synthase kinase 3. Genes Dev. 10:1443-1454.

10. Yang, S. D., J. S. Song, J. S. Yu, and S. G. Shiah. 1993. Protein kinase B/GSK-3 phosphorylates tau on Ser235-Pro and Ser404-Pro that are abnormally phosphorylated in Alzheimer's disease brain. J. Neurochem. 61:1742-1747.

11. Yu, J. S., and S. D. Yang. 1994. Protein kinase B/glycogen synthase kinase-3 predominantly phosphorylates the in vivo site Thr97-Pro in brain myelin basic protein: Evidence for Thr-Pro and Ser-Arg-X- X-Ser as consensus sequence motifs. J. Neurochem. 62:1596-1603.

12. Diehl, J. A., M. Cheng, M. F. Roussel, and C. J. Sherr. 1998. Glycogen synthase kinase-3b regulates cyclin D1 proteolysis and subcellular localization. Genes Dev. 12:3499-3511.

13. Morisco, C., K. Seta, S. E. Hardt, Y. Lee, S. F. Vatner, and J. Sadoshima. 2001. Glycogen synthase kinase 3b regulates GATA4 in cardiac myocytes. J. Biol. Chem. 276:28586-28597.

14. Boyle, W. J., T. Smeal, L. H. Defize, P. Angel, J. R. Woodgett, M. Karin, and T. Hunter. 1991. Activation of protein kinase C decreases phosphorylation of c-Jun at sites that negatively regulate its DNA-binding activity. Cell. 64:573-584.

15. Plyte, S. E., K. Hughes, E. Nikolakaki, B. J. Pulverer, and J. R. Woodgett. 1992. Glycogen synthase kinase-3: functions in oncogenesis and development. Biochim. Biophys. Acta. 1114:147-162.

16. Fiol, C. J., J. S. Williams, C. H. Chou, Q. M. Wang, P. J. Roach, and O. M. Andrisani. 1994. A secondary phosphorylation of CREB³⁴¹ at Ser¹²⁹ is required for the cAMP-mediated control of gene expression. J. Biol. Chem. 269:32187-32193.

17. Welsh, G. I., and C. G. Proud. 1993. Glycogen synthase kinase-3 is rapidly inactivated in response to insulin and phosphorylates eukaryotic initiation factor eIF-2B. Biochem. J. 294:625-629.

18. Chu, B., F. Soncin, B. D. Price, M. A. Stevenson, and S. K. Calderwood. 1996. Sequential phosphorylation by mitogen-activated protein kinase and glycogen synthase kinase 3 represses transcriptional activation by heat shock factor-1. J. Biol. Chem. 271:30847-30857.

19. Turenne, G. A., and B. D. Price. 2001. Glycogen synthase kinase3 beta phosphorylates serine 33 of p53 and activates p53's transcriptional activity. B.M C. Cell Biol. 2:12.

20. Weston, C. R., and R. J. David. 2001. Signaling specificity—a complex affair. Science. 292:2439-2440.

21. Sutherland, C., I. A. Leighton, and P. Cohen. 1993. Inactivation of glycogen synthase kinase-3 beta by phosphorylation: New kinase connections in insulin and growth-factor signaling. Biochem. J 296:15-19.

22. Cross, D. A., D. R. Alessi, J. R. Vandenheede, H. E. McDowell, H. S. Hundal, and P. Cohen. 1994. The inhibition of glycogen synthase kinase-3 by insulin or insulin-like growth factor 1 in the rat skeletal muscle cell line L6 is blocked by wortmannin, but not by rapamycin: Evidence that wortmannin blocks activation of the mitogen-activated protein kinase pathway in L6 cells between Ras and Raf. Biochem. J 303:21-26.

23. Delcommenne, M., C. Tan, V. Gray, L. Rue, J. Woodgett, and S. Dedhar. 1998. Phosphoinositide-3-OH kinase-dependent regulation of glycogen synthase kinase 3 and protein kinase B/Akt by integrin-linked kinase. Proc. Natl. Acad. Sci. USA. 95:11211-11216.

24. Li, M., X. Wang, M. K. Meintzer, T. Laessig, M. J. Birnbaum, and K. A. Heidenreich. 2000. Cyclic AMP promotes neuronal survival by phosphorylation of glycogen synthase kinase 3b. Mol. Cell. Biol. 20:9356-9363.

25. Ruel, L., V. Stambolic, A. Ali, A. S. Manoukian, and J. R. Woodgett. 1999. Regulation of the protein kinase activity of Shaggy(Zeste-white3) by components of the wingless pathway in Drosophila cells and embryos. J. Biol. Chem. 274:21790-21796.

26. Ferkey, D. M., and D. Kimelman. 2000. GSK-3: New thoughts on an old enzyme. Dev. Biol. 225:471-479.

27. Haq, S., G. Choukroun, Z. B. Kang, H. Ranu, T. Matsui, A. Rosenzweig, J. D. Molkentin, A. Alessandrini, J. Woodgett, R. Hajjar, A. Michael, and T. Force. 2000. Glycogen synthase kianse-3b is a negative regulator of cardiomyocyte hypertrophy. J. Cell Biol. 151:117-129.

28. Hall, J. L., J. C. Chatham, H. Eldar-Finkelman, and G. H. Gibbons. 2001. Upregulation of glucose metabolism during intimal lesion formation is coupled to the inhibition of vascular smooth muscle cell apoptosis; role of GSK3b. Diabetes. 50:1171-1179.

29. Jaffe, E. A., R. L. Nachman, C. G. Becker, and C. R. Minick. 1973. Culture of human endothelial cells derived from umbilical veins. Identification by morphologic and immunologic criteria. J Clin Invest. 52:2745-2756.

30. Summers, S. A., A. W. Kao, A. D. Kohn, G. S. Backus, R. A. Roth, J. E. Pessin, and M. J. Birnbaum. 1999. The role of glycogen synthase kinase 3b in insulin-stimulated glucose metabolism. J. Biol. Chem. 274:17934-17940.

31. Eldar-Finkelman, H., G. M. Argast, O. Foord, E. H. Fischer, and E. G. Krebs. 1996. Expression and characterization of glycogen synthase kinase-3 mutants and their effect on glycogen synthase activity in intact cells. Proc. Natl. Acad Sci. USA. 93:10228-10233.

32. Smith, R. C., D. Branellec, D. H. Gorski, K. Guo, H. Perlman, J.-F. Dedieu, C. Pastore, A. Mahfoudi, P. Denefle, J. M. Isner, and K. Walsh. 1997. p21^{CIP1}-mediated inhibition of cell proliferation by overexpression of the gax homeodomain gene. Genes Dev. 11:1674-1689.

33. Frisch, S. M., and H. Francis. 1994. Disruption of epithelial cell-matrix interactions induces apoptosis. J. Cell Biol. 124:619-626.

34. Witzenbichler, B., Y. Kureishi, Z. Luo, A. Le Roux, D. Branellec, and K. Walsh. 1999. Regulation of smooth muscle cell migration and integrin expression by the Gax transcription factor. J. Clin. Invest. 104:1469-1480.

35. Passaniti, A., R. M. Taylor, R. Pili, Y. Guo, P. V. Long, J. A. Haney, R. R. Pauly, D. S. Grant, and G. R. Martin. 1992. Methods in laboratory investigation: a simple, quantitative method for assessing angiogenesis and antiangiogenic agents using reconstituted basement membrane, heparin, and fibroblast growth factor. Lab. Invest. 67:519-528.

36. Muhlhauser, J., M. J. Merrill, R. Pili, H. Maeda, M. Bacic, B. Bewig, A. Passaniti, N. A. Edwards, R. G. Crystal, and M. D. Capogrossi. 1995. VEGF₁₆₅ expressed by a replication-deficient recombinant adenovirus vector induces angiogenesis in vivo. Circ. Res. 77:1077-1086.

37. Nakao, T., M. Abe, K. Tanaka, R. Shineha, S. Satomi, and Y. Sato. 2000. Angiogenesis inhibition by transdominant mutant Ets-1. J. Cell Physiol. 184:255-262.

38. Fujio, Y., and K. Walsh. 1999. Akt mediates cytoprotection of endothelial cells by vascular endothelial growth factor in an anchorage-dependent manner. J. Biol. Chem. 274:16349-16354.

39. Brooks, P. C., A. M. P. Montgomery, M. Rosenfeld, R. A. Reisfeld, T. Hu, G. Klier, and D. A. Cheresh. 1994. Integrin aᵥb₃ antagonists promote tumor regression by inducing apoptosis of angiogenic blood vessels. Cell:1 157-1164.

40. Alon, T., I. Hemo, A. Itin, J. Pe'er, J. Stone, and E. Keshet. 1995. Vascular endothelial growth factor acts as a survival factor for newly formed retinal vessels and has implications for retinopathy of prematurity. Nat. Med 1:1024-1028.

41. Yuan, F., Y. Chen, M. Dellian, N. Safabakhsh, N. Ferrara, and R. K. Jain. 1996. Time-dependent vascular regression and permeability changes in established human tumor xenografts induced by an anti-vascular endothelial growth factor/vascular permeability factor antibody. Proc. Natl. Acad. Sci. USA. 93:14765-14770.

42. Benjamin, L. E., and E. Keshet. 1997. Conditional switching of vascular endothelial growth factor (VEGF) expression in tumors: induction of endothelial cell shedding and regression of hemangioblastoma-like vessels by VEGF withdrawal. Proc. Natl. Acad Sci. USA. 94:8761-8766.

43. Gerber, H. -P., A. McMurtrey, J. Kowalski, M. Yan, B. A. Key, V. Dixit, and N. Ferrara. 1998. Vascular endothelial growth factor regulates endothelial cell survival through the phosphatidylinositol 3′-kinase/Akt signal transduction pathway: Requirement for FIk-1/KDR activation. J. Biol. Chem. 273:30336-30343.

44. Papapetropoulos, A., D. Fulton, K. Mahboubi, R. G. Kalb, D. S. O'Connor, F. Li, D. C. Altieri, and W. C. Sessa. 2000. Angiopoietin-1 inhibits endothelial cell apoptosis via the Akt/Survivin pathway. J. Biol. Chem. 275:9102-9105.

45. Kim, I., H. G. Kim, J. -N. So, J. H. Kim, H. J. Kwak, and G. Y. Koh. 2000. Angiopoietin-1 regulates endothelial cell survival through the phosphatidylinositol 3′-kinase/Akt signal transduction pathway. Circ. Res. 86:24-29.

46. Hannigan, G. E., C. Leung-Hagesteijn, L. Fitz-Gibbon, M. G. Coppolino, G. Radeva, J. Filmus, J. C. Bell, and S. Dedhar. 1996. Regulation of cell adhesion and anchorage-dependent growth by a new b1-integrin-linked protein kinase. Nature. 379:91-96.

47. Troussard, A. A., C. Tan, T. N. Yoganathan, and S. Dedhar. 1999. Cell-extracellular matrix interactions stimulate the AP-1 transcription factor in an integrin-linked kinase- and glycogen synthase kinase 3-dependent manner. Mol. Cell. Biol. 19:7420-7427.

48. Morales-Ruiz, M., G. Fulton, G. Sowa, L. R. Languino, Y. Fujio, K. Walsh, and W. C. Sessa. 2000. Vascular endothelial growth factor-stimulated actin reorganization and migration of endothelial cells is regulated via the serine/threonine kinase Akt. Circ. Res. 86:892-896.

49. Fulton, D., J. -P. Gratton, T. J. McCabe, J. Fontana, Y. Fujio, K. Walsh, T. F. Franke, A. Papapetropoulos, and W. C. Sessa. 1999. Regulation of endothelium-derived nitric oxide production by the protein kinase Akt. Nature. 399:597-601.

50. Luo, Z., Y. Fujio, Y. Kureishi, R. D. Rudic, G. Daumerie, D. Fulton, W. C. Sessa, and K. Walsh. 2000. Acute modulation of endothelial Akt/PKB activity alters nitric oxide-dependent vasomotor activity in vivo. J. Clin. Invest. 106:493-499.

51. Kureishi, Y., Z. Luo, I. Shiojima, A. Bialik, D. Fulton, D. J. Lefer, W. C. Sessa, and K. Walsh. 2000. The HMG-CoA reductase inhibitor simvastatin activates the protein kinase Akt and promotes angiogenesis in normocholesterolemic animals. Nat. Med. 6:1004-1010.

52. Duplaa, C., B. Jaspard, C. Moreau, and P. A. D'Amore. 1999. Identification and cloning of a secreted protein related to the cysteine-rich domain of frizzled. Evidence for a role in endothelial cell growth control. Circ. Res. 84:1433-1445.

All references disclosed herein are incorporated by reference in their entirety.

We claim:

1. A method for inhibiting angiogenesis comprising:
administering to a subject in need of such treatment an angiogenesis inhibitor selected from the group consisting of: (1) an active glycogen synthase kinase-3 (GSK3) molecule, and (2) a GSK3 kinase activator;
wherein the angiogenesis inhibitor is administered in an amount effective to inhibit angiogenesis in the subject.

2. The method of claim 1, wherein the subject has a condition associated with excessive endothelial cell proliferation.

3. The method of claim 1, wherein the subject does not have a condition otherwise calling for treatment with an Akt inhibitor or an agent that downregulates expression of an Akt molecule in the subject.

4. The method of claim 1, wherein the angiogenesis inhibitor is administered acutely.

5. The method of claim 1, wherein the angiogenesis inhibitor is an active GSK3 molecule.

6. The method of claim 5, wherein the active GSK3 molecule is an active GSK3 nucleic acid molecule.

7. The method of claim 5, wherein the active GSK3 molecule is an active GSK3 polypeptide molecule.

8. The method of claim 1, wherein the angiogenesis inhibitor is a GSK3 kinase activator.

9. The method of claim 1, wherein the angiogenesis inhibitor is a GSK3 kinase activator that induces or maintains an active conformation in a GSK3 protein.

10. A method for enhancing angiogenesis comprising:
administering to a subject in need of such treatment an angiogenesis promoter selected from the group consisting of: (1) an inactive GSK3 molecule, and (2) a GSK3 kinase inhibitor;
wherein the angiogenesis promoter is administered in an amount effective to enhance angiogenesis in the subject.

11. The method of claim 10, wherein the subject has or is at risk of having a condition selected from the group consisting of: myocardial infarction, ischemia-reperfusion injury, dilated cardiomyopathy, and conductive system disorders.

12. The method of claim 10, wherein the subject does not have a condition otherwise calling for treatment with an Akt molecule or an agent that upregulates expression of an Akt molecule in the subject.

13. The method of claim 10, wherein the angiogenesis promoter is administered acutely.

14. The method of claim 10, wherein the angiogenesis promoter is an inactive GSK3 molecule.

15. The method of claim 14, wherein the inactive GSK3 molecule is an inactive GSK3 nucleic acid molecule.

16. The method of claim 14, wherein the inactive GSK3 molecule is an inactive GSK3 polypeptide molecule.

17. The method of claim 10, wherein the angiogenesis promoter is a GSK3 kinase inhibitor.

18. The method of claim 10, wherein the angiogenesis promoter is a GSK3 kinase inhibitor selected from the group consisting of: a substrate analog and an allosteric effector analog.

19. The method of claim 10 further comprising co-administering one or more anti-arteriosclerotic agent(s) selected from the group consisting of: 3-hydroxy-3-methylglutaryl-coenzyme A (HMG-CoA) reductase inhibitor, a diuretic, an antiadrenergic agent, a vasodilator, a calcium channel antagonist, an angiotensin-converting enzyme (ACE) inhibitor, an angiotensin II antagonist, and a clot dissolver.

20. A method for inhibiting an endothelial cell activity, comprising:
contacting an endothelial cell with an angiogenesis inhibitor in an effective amount to inhibit an endothelial cell activity.

21. The method of claim 20, wherein the endothelial cell activity is endothelial cell viability.

22. The method of claim 20, wherein the endothelial cell activity is endothelial cell migration.

23. The method of claim 20, wherein the endothelial cell activity is angiogenesis.

24. The method of claim 20, wherein the contacting is performed in vitro.

25. The method of claim 20, wherein the contacting is performed in vivo.

26. The method of claim 20, wherein the angiogenesis inhibitor is an active GSK3 molecule.

27. The method of claim 26, wherein the active GSK3 molecule is an active GSK3 nucleic acid molecule.

28. The method of claim 26, wherein the active GSK3 molecule is an active GSK3 polypeptide molecule.

29. The method of claim 20, wherein the angiogenesis inhibitor is a GSK3 kinase activator.

30. The method of claim 20, wherein the angiogenesis inhibitor is a GSK3 kinase activator that induces or maintains an active conformation in a GSK3 polypeptide.

31. A method for enhancing an endothelial cell activity, comprising:
contacting an endothelial cell with an angiogenesis promoter in an effective amount to enhance an endothelial cell activity.

32. The method of claim 31, wherein the endothelial cell activity is endothelial cell viability.

33. The method of claim 31, wherein the endothelial cell activity is endothelial cell migration.

34. The method of claim 31, wherein the endothelial cell activity is angiogenesis.

35. The method of claim 31, wherein the contacting is performed in vitro.

36. The method of claim 31, wherein the contacting is performed in vivo.

37. The method of claim 31, wherein the angiogenesis promoter is an inactive GSK3 molecule.

38. The method of claim 37, wherein the inactive GSK3 molecule is an inactive GSK3 nucleic acid molecule.

39. The method of claim 37, wherein the inactive GSK3 molecule is an inactive GSK3 polypeptide molecule.

40. A method for inhibiting apoptotic cell death of an endothelial cell, comprising:
contacting an angiogenesis promoter with an endothelial cell
wherein the angiogenesis promoter is present in an amount effective to inhibit apoptotic cell death of the endothelial cell.

41. The method of claim 40 wherein the endothelial cell is a vascular endothelial cell.

42. The method of claim 40 wherein the angiogenesis promoter is an inactive GSK3 molecule.

43. The method of claim 42, wherein the inactive GSK3 molecule is an inactive GSK3 nucleic acid molecule.

44. The method of claim 42, wherein the inactive GSK3 molecule is an inactive GSK3 polypeptide molecule.

45. The method of claim 40 wherein the angiogenesis promoter is a GSK3 kinase inhibitor.

46. The method of claim 40, wherein the endothelial cell is part of a tissue or an organ to be transplanted.

47. The method of claim 40, wherein the contacting of an angiogenesis promoter with an endothelial cell comprises acute administration of the angiogenesis promoter.

48 The method of claim 40, wherein the contacting of an angiogenesis promoter with an endothelial cell comprises prophylactic administration of the angiogenesis promoter.

49. The method of any one of claims 40-48, further comprising co-administering a growth factor.

50. The method of claim 49, wherein the growth factor is a vascular endothelial growth factor (VEGF).

51. The method of claim 40, wherein the angiogenesis promoter is an inactive GSK3 molecule.

52. The method of claim 51, wherein the inactive GSK3 molecule is an inactive GSK3 nucleic acid molecule.

53. The method of claim 51, wherein the inactive GSK3 molecule is an inactive GSK3 polypeptide molecule.

54. The method of claim 40, wherein the angiogenesis promoter is a GSK3 kinase inhibitor.

55. A method of screening for a GSK3 kinase activator that inhibits an endothelial cell activity, comprising:
contacting a test molecule with an endothelial cell and
determining whether the test molecule inhibits an endothelial cell activity;
wherein an inhibition of an endothelial cell activity in the presence of the test molecule indicates that the test molecule is a GSK3 kinase activator.

56. The method of claim 55, wherein determining whether the test molecule inhibits an endothelial cell activity further includes determining whether the test molecule binds a GSK3 molecule.

57. A method of screening for a GSK3 kinase inhibitor that enhances an endothelial cell activity, comprising:
contacting a test molecule with an endothelial cell and
determining whether the test molecule enhances an endothelial cell activity;
wherein an enhancement of an endothelial cell activity in the presence of the test molecule indicates that the test molecule is a GSK3 kinase inhibitor.

58. The method of claim 57, wherein determining whether the test molecule enhances an endothelial cell activity further includes determining whether the test molecule binds a GSK3 molecule.

59. The method of any one of claims 55-58, wherein the contacting is performed in vitro.

60. The method of any one of claims 55-58, wherein the contacting is performed in vivo.

61. The method of any one of claims 55-58, wherein the test molecule is obtained from a library of molecules.

62. A method for treating a condition associated with increased apoptotic cell death of vascular endothelial cells, comprising:
administering to a subject in need of such treatment an angiogenesis promoter in an amount effective to inhibit increased apoptotic cell death of the vascular endothelial cells.

63. The method of claim 62, wherein the angiogenesis promoter is an inactive GSK3 molecule

64. The method of claim 63, wherein the inactive GSK3 molecule is an inactive GSK3 nucleic acid molecule.

65. The method of claim 63, wherein the inactive GSK3 molecule is an inactive GSK3 polypeptide molecule.

66. The method of claim 62, wherein the condition is characterized by a lesion of a blood vessel wall.

67. The method of claim 62, wherein the subject is hyperlipidemic.


SEQUENCE LISTING (14 sequences)

SEQ ID NO: 1 - DNA, 651 bp, Homo sapiens
ttacaggtgtgagccacctcgcccagctgagttcagtataattttcaatgagaaactgaaattcagttttataatcaaagagcatgtttgctgaagccatcattctcagcaaactaatacagggacagaaaaccaaacaccgcatgttccactcataagtgggagttgaacaatgagaacacacggacacagggagggaaacatcacacaccagggcctgtcaggcggtcaggggtaaggggagagagagcatcgagacaaatatctaaggtatgcggggcttaaaacctagatgatggttgataggtgcagcaaaccaccatggcacatgtatacctgtgtaacaaacccgcacgtcctgcacatgcatcccacaacttaaagcaaaataaaaatatatatatttttcatattttcatatataatatataaatatataattaagataaaatattacatattacatatgtataaattcatatataacatataaaatatataatattatatattatatacatgtgtatataaaatctggctgcggagtttttgatctatacattgaacaaattgtctcacctactgatgaaaaggtgattcgcgaagagagtgatcatgtcagggcggcccagaaccacctcctttgcggag

SEQ ID NO: 2 - protein, 420 aa, Homo sapiens
MSGRPRTTSFAESCKPVQQPSAFGSMKVSRDKDGSKVTTVVATPGQGPDRPQEVSYTDTKLIGNGSFGVVYQAKLCDSGELVAIKKVLQDKRFKNRELQIMRKLDHCNIVRLRYFFYSSGEKKDEVYLNLVLDYVPETVYRVARHYSRAKQTLPVIYVKLYMYQLFRSLAYIHSFGICHRDIKPQNLLLDPDTAVLKLCDFGSAKQLVRGEPNVSYICSRYYRAPELIFGATDYTSSIDVWSAGCVLAELLLGQPIFPGDSGVDQLVEIIKVLGTPTREQIREMNPNYTEFKFPQIKAHPWTKVFRPRTPPEAIALCSRLLEYTPTARLTPLEACAHSFFDELRDPNVKLPNGRDTPALFNFTTQELSSNPPLATILIPPHARIQAAASTPTNATAASDANTGDRGQTNNAASASASNST

SEQ ID NO: 3 - protein, 483 aa, Homo sapiens
MSGGGPSGGGPGGSGRARTSSFAEPGGGGGGGGGGPGGSASGPGGTGGGKASVGAMGGGVGASSSGGGPGGSGGGGSGGPGAGTSFPPPGVKLGRDSGKVTTVVATLGQGPERSQEVAYTDIKVIGNGSFGVVYQARLAETRELVAIKKVLQDKRFKNRELQIMRKLDHCNIVRLRYFFYSSGEKKDELYLNLVLEYVPETVYRVARHFTKAKLTIPILYVKVYMYQLFRSLAYIHSQGVCHRDIKPQNLLVDPDTAVLKLCDFGSAKQLVRGEPNVSYICSRYYRAPELIFGATDYTSSIDVWSAGCVLAELLLGQPIFPGDSGVDQLVEIIKVLGTPTREQIREMNPNYTEFKFPQIKAHPWTKVFKSRTPPEAIALCSSLLEYTPSSRLSPLEACAHSFFDELRCLGTQLPNNRPLPPLFNFSAGELSIQPSLNAILIPPHLRSPAGTTTLTPSSQALTETPTSSDWQSTDATPTLTNSS

SEQ ID NO: 4 - protein, 483 aa, Homo sapiens
MSGGGPSGGGPGGSGRARTSSFAEPGGGGGGGGGGPGGSASGPGGTGGGKASVGAMGGGVGASSSGGGPGGSGGGGSGGPGAGTSFPPPGVKLGRDSGKVTTVVATLGQGPERSQEVAYTDIKVIGNGSFGVVYQARLAETRELVAIKKVLQDKRFKNRELQIMRKLDHCNIVRLRYFFYSSGEKKDELYLNLVLEYVPETVYRVARHFTKAKLTIPILYVKVYMYQLFRSLAYIHSQGVCHRDIKPQNLLVDPDTAVLKLCDFGSAKQLVRGEPNVSYICSRYYRAPELIFGATDYTSSIDVWSAGCVLAELLLGQPIFPGDSGVDQLVEIIKVLGTPTREQIREMNPNYTEFKFPQIKAHPWTKVFKSRTPPEAIALCSSLLEYTPSSRLSPLEACAHSFFDELRCLGTQLPNNRPLPPLFNFSAGELSIQPSLNAILIPPHLRSPSGTTTLTPSSQALTETPTSSDWQSTDATPTLTNSS

SEQ ID NO: 5 - protein, 483 aa, Homo sapiens; identical to SEQ ID NO: 3

SEQ ID NO: 6 - protein, 420 aa, Homo sapiens
MSGRPRTTSFAESCKPVQQPSAFGSMKVSRDKDGSKVTTVVATPGQGPDRPQEVSYTDTKVIGNGSFGVVYQAKLCDSGELVAIKKVLQDKRFKNRELQIMRKLDHCNIVRLRYFFYSSGEKKDEVYLNLVLDYVPETVYRVARHYSRAKQTLPVIYVKLYMYQLFRSLAYIHSFGICHRDIKPQNLLLDPDTAVLKLCDFGSAKQLVRGEPNVSYICSRYYRAPELIFGATDYTSSIDVWSAGCVLAELLLGQPIFPGDSGVDQLVEIIKVLGTPTREQIREMNPNYTEFKFPQIKAHPWTKVFRPRTPPEAIALCSRLLEYTPTARLTPLEACAHSFFDELRDPNVKLPNGRDTPALFNFTTQELSSNPPLATILIPPHARIQAAASTPTNATAASDANTGDRGQTNNAASASASNST

SEQ ID NO: 7 - protein, 420 aa, Homo sapiens
MSGRPRTTSFAESCKPVQQPSAFGSMKVSRDKDGSKVTTVVATPGQGPDRPQEVSYTDTKVIGNGSFGVVYQAKLCDSGELVAIKKVLQDKRFKNRELQIMRKLDHCNIVRLRYFFYSSGEKKDEVYLNLVLDYVPETVYRVARHYSRAKQTLPVIYVKLYMYQLFRSLAYIHSFGICHRDIKPQNLLLDPDTAVLKLCDFGSAKQLVRGEPNVSYICSRYYRAPELIFGATDYTSSIDVWSAGCVLAELLLGQPIFPGDSGVDQLVEIIKVLGTPTREQIREMNPNYTEFKFPQIKAHPWTKVFRPRTPPEAIALCSRLLEYTPTARLTPLEACAHSFFDELRDPNVKHPNGRDTPALFNFTTQELSSNPPLATILIPPHARIQAAASTPTNATAASDANTGDRGQTNNAASASASNST

SEQ ID NO: 8 - protein, 433 aa, Homo sapiens
MSGRPRTTSFAESCKPVQQPSAFGSMKVSRDKDGSKVTTVVATPGQGPDRPQEVSYTDTKVIGNGSFGVVYQAKLCDSGELVAIKKVLQDKRFKNRELQIMRKLDHCNIVRLRYFFYSSGEKKDEVYLNLVLDYVPETVYRVARHYSRAKQTLPVIYVKLYMYQLFRSLAYIHSFGICHRDIKPQNLLLDPDTAVLKLCDFGSAKQLVRGEPNVSYICSRYYRAPELIFGATDYTSSIDVWSAGCVLAELLLGQPIFPGDSGVDQLVEIIKVLGTPTREQIREMNPNYTEFKFPQIKAHPWTKDSSGTGHFTSGVRVFRPRTPPEAIALCSRLLEYTPTARLTPLEACAHSFFDELRDPNVKLPNGRDTPALFNFTTQELSSNPPLATILIPPHARIQAAASTPTNATAASDANTGDRGQTNNAASASASNST

SEQ ID NO: 9 - DNA, 2170 bp, Homo sapiens
gccagagcggcgcggcctggaagaggccagggcccgggggaggcggcggcagcggcggcggctggggcagcccgggcagcccgagccccgcagcctgggcctgtgctcggcgccatgagcggcggcgggccttcgggaggcggccctgggggctcgggcagggcgcggactagctcgttcgcggagcccggcggcggaggcggaggaggcggcggcggccccggaggctcggcctccggcccaggcggcaccggcggcggaaaggcatctgtcggggccatgggtgggggcgtcggggcctcgagctccgggggtggacccggcggcagcggcggaggaggcagcggaggccccggcgcaggcactagcttcccgccgcccggggtgaagctgggccgtgacagcgggaaggtgaccacagtcgtagccactctaggccaaggcccagagcgctcccaagaagtggcttacacggacatcaaagtgattggcaatggctcatttggggtcgtgtaccaggcacggctggcagagaccagggaactagtcgccatcaagaaggttctccaggacaagaggttcaagaaccgagagctgcagatcatgcgtaagctggaccactgcaatattgtgaggctgagatactttttctactccagtggcgagaagaaagacgagctttacctaaatctggtgctggaatatgtgcccgagacagtgtaccgggtggcccgccacttcaccaaggccaagttgaccatccctatcctctatgtcaaggtgtacatgtaccagctcttccgcagcttggcctacatccactcccagggcgtgtgtcaccgcgacatcaagccccagaacctgctggtggaccctgacactgctgtcctcaagctctgcgattttggcagtgcaaagcagttggtccgaggggagcccaatgtctcctacatctgttctcgctactaccgggccccagagctcatctttggagccactgattacacctcatccatcgatgtttggtcagctggctgtgtactggcagagctcctcttgggccagcccatcttccctggggacagtggggtggaccagctggtggagatcatcaaggtgctgggaacaccaacccgggaacaaatccgagagatgaaccccaactacacggagttcaagttccctcagattaaagctcacccctggacaaaggtgttcaaatctcgaacgccgccagaggccatcgcgctctgctctagcctgctggagtacaccccatcctcaaggctctccccactagaggcctgtgcgcacagcttctttgatgaactgcgatgtctgggaacccagctgcctaacaaccgcccacttccccctctcttcaacttcagtgctggtgaactctccatccaaccgtctctcaacgccattcttatccctcctcacttgaggtccccagcgggcactaccaccctcaccccgtcctcacaagctttaactgagactccgaccagctcagactggcagtcgaccgatgccacacctaccctcactaactcctcctgagggccccaccaagcacccttccacttccatctgggagccccaagaggggctgggaaggggggccatagcccatcaagctcctgccctggctgggcccctagactagagggcagaggtaaatgagtccctgtccccacctccagtccctccctcaccagcctcacccctgtggtgggctttttaagaggattttaactggttgtggggagggaagagaaggacagggtgttggggggatgaggacctcctacccccttggccccctcccctcccccagacctccacctcctccagaccccctcccctcctgtgtcccttgtaaatagaaccagcccagcccgtctcctcttcccttccctggcccccgggtgtaaatagattgttataatttttttcttaaagaaaacgtcgattcgcaccgtccaacctggccccgcccctcctacagctgtaactcccctcctgtcctctgcccccaaggtctactccctcctcaccccaccctggagggccaggggagtggagagagctcctgatgtcttagtttccacagtaaggtttgcctgtgtacagacctccgttcaataaattattggcatgaaaa

SEQ ID NO: 10 - protein, 483 aa, Homo sapiens; identical to SEQ ID NO: 3

SEQ ID NO: 11 - DNA, 2169 bp, Homo sapiens
gccagagcggcgcggcctggaagaggccagggcccgggggaggcgacggcagcggcggcggctggggcagcccgggcagcccgagccccgcagcctgggcctgtgctcggcgccatgagcggcggcgggccttcgggaggcggccctgggggctcgggcagggcgcggactagctcgttcgcggagcccggcggcggaggcggaggaggcggcggcggccccggaggctcggcctccggcccaggcggcaccggcggcggaaaggcatctgtcggggccatgggtgggggcgtcggggcctcgagctccgggggtggacccggcggcagcggcggaggaggcagcggaggccccggcgcaggcactagcttcccgccgcccggggtgaagctgggccgtgacagcgggaaggtgaccacagtcgtagccactctaggccaaggcccagagcgctcccaagaagtggcttacacggacatcaaagtgattggcaatggctcatttggggtcgtgtaccaggcacggctggcagagaccagggaactagtcgccatcaagaaggttctccaggacaagaggttcaagaaccgagagctgcagatcatgcgtaagctggaccactgcaatattgtgaggctgagatactttttctactccagtggcgagaagaaagacgagctttacctaaatctggtgctggaatatgtgcccgagacagtgtaccgggtggcccgccacttcaccaaggccaagttgaccatccctatcctctatgtcaaggtgtacatgtaccagctcttccgcagcttggcctacatccactcccagggcgtgtgtcaccgcgacatcaagccccagaacctgctggtggaccctgacactgctgtcctcaagctctgcgattttggcagtgcaaagcagttggtccgaggggagcccaatgtctcctacatctgttctcgctactaccgggccccagagctcatctttggagccactgattacacctcatccatcgatgtttggtcagctggctgtgtactggcagagctcctcttgggccagcccatcttccctggggacagtggggtggaccagctggtggagatcatcaaggtgctgggaacaccaacccgggaacaaatccgagagatgaaccccaactacacggagttcaagttccctcagattaaagctcacccctggacaaaggtgttcaaatctcgaacgccgccagaggccatcgcgctctgctctagcctgctggagtacaccccatcctcaaggctctccccactagaggcctgtgcgcacagcttctttgatgaactgcgatgtctgggaacccagctgcctaacaaccgcccacttccccctctcttcaacttcagtgctggtgaactctccatccaaccgtctctcaacgccattctcatccctcctcacttgaggtcccccagcggcactaccaccctcaccccgtcctcacaagctttaactgagactccgaccagctcagactggcagtcgaccgatgccacacctaccctcactaactcctcctgagggccccaccaagcacccttccacttccatctgggagccccaagagggcgtgggaaggggggccatagcccatcaagctcctgccctggctgggcccctagactagagggcagaggtaaatgagtccctgtccccacctccagtccctccctcaccagcctcacccctgtggtgggctttttaagaggattttaactggttgtggggagggaagagaaggacagggtgttggggggatgaggacctcctacccccttggccccctcccctcccccagacctccacctcctccagaccccctcccctcctgtgtcccttgtaaatagaaccagcccagcccgtctcctcttcccttccctggcccccgggtgtaaatagattgttataatttttttcttaaagaaaacgtcgattcgcaccgtccaacctgccccgcccctcctacagctgtaactcccctcctgtcctctgcccccaaggtctactccctcctcaccccaccctggagggccaggggagtggagagagctcctgatgtcttagtttccacagtaaggtttgcctgtgtacagacctccgttcaataaattattggcatgaaaa

SEQ ID NO: 12 - protein, 483 aa, Homo sapiens; identical to SEQ ID NO: 4

SEQ ID NO: 13 - DNA, 1639 bp, Homo sapiens
atcatctatatgttaaatatccgtgccgatctgtcttgaaggagaaatatatcgcttgttttgttttttatagtatacaaaaggagtgaaaagccaagaggacgaagtctttttctttttcttctgtgggagaacttaatgctgcatttatcgttaacctaacaccccaacataaagacaaaaggaagaaaaggaggaaggaaggaaaaggtgattcgcgaagagagtgatcatgtcagggcggcccagaaccacctcctttgcggagagctgcaagccggtgcagcagccttcagcttttggcagcatgaaagttagcagagacaaggacggcagcaaggtgacaacagtggtggcaactcctgggcagggtccagacaggccacaagaagtcagctatacagacactaaagtgattggaaatggatcatttggtgtggtatatcaagccaaactttgtgattcaggagaactggtcgccatcaagaaagtattgcaggacaagagatttaagaatcgagagctccagatcatgagaaagctagatcactgtaacatagtccgattgcgttatttcttctactccagtggtgagaagaaagatgaggtctatcttaatctggtgctggactatgttccggaaacagtatacagagttgccagacactatagtcgagccaaacagacgctccctgtgatttatgtcaagttgtatatgtatcagctgttccgaagtttagcctatatccattcctttggaatctgccatcgggatattaaaccgcagaacctcttgttggatcctgatactgctgtattaaaactctgtgactttggaagtgcaaagcagctggtccgaggagaacccaatgtttcgtatatctgttctcggtactatagggcaccagagttgatctttggagccactgattatacctctagtatagatgtatggtctgctggctgtgtgttggctgagctgttactaggacaaccaatatttccaggggatagtggtgtggatcagttggtagaaataatcaaggtcctgggaactccaacaagggagcaaatcagagaaatgaacccaaactacacagaatttaaattccctcaaattaaggcacatccttggactaaggattcgtcaggaacaggacatttcacctcaggagtgcgggtcttccgaccccgaactccaccggaggcaattgcactgtgtagccgtctgctggagtatacaccaactgcccgactaacaccactggaagcttgtgcacattcattttttgatgaattacgggacccaaatgtcaaactaccaaatgggcgagacacacctgcactcttcaacttcaccactcaagaactgtcaagtaatccacctctggctaccatccttattcctcctcatgctcggattcaagcagctgcttcaacccccacaaatgccacagcagcgtcagatgctaatactggagaccgtggacagaccaataatgctgcttctgcatcagcttccaactccacctgaacagtcccgagcagccagctgcacaggaaaaaccaccagttacttgagtgtcactcagcaacactggtcacgtttggaaagaatattaaaaaaaaaaaaaaaaaa

SEQ ID NO: 14 - protein, 433 aa, Homo sapiens; identical to SEQ ID NO: 8